(19) United States
(12) Patent Application Publication
(10) Pub. No.: US 2003/0082591 A1
(43) Pub. Date: May 1, 2003

(54) Methods for gene disruption and uses thereof

(76) Inventors: Donald Awrey, Mississauga (CA); Jack Greenblatt, Toronto (CA)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/202,442

(22) Filed: Jul. 24, 2002

Related U.S. Application Data
(60) Provisional application No. 60/307,461, filed on Jul. 24, 2001.

(51) Int. Cl. C12Q 1/68; G06F 19/00; G01N 33/48; G01N 33/50; C12N 15/87; C12N 15/74; C12N 1/21; C12N 5/06

(52) U.S. Cl. 435/6; 435/455; 435/471; 435/252.3; 435/325; 702/20

(57) ABSTRACT

The present invention relates to compositions and methods for in-frame disruption of a gene sequence by homologous recombination. The present invention may be used in certain embodiments to disrupt a gene without causing any downstream effects on non-target sequences. In certain embodiments, the inventive methods may be used to identify and/or characterize products encoded by essential genes, conditionally essential genes, and non-essential genes.

### RELATED APPLICATION INFORMATION

This application claims the benefit of priority to Provisional Patent Application No. 60/307,461, filed Jul. 24, 2001, which application is hereby incorporated by reference in its entirety.

### INTRODUCTION

An analysis of the discovery of novel antimicrobial agents illustrates the problems researchers in all fields of drug development face today. The increasing prevalence of drug-resistant pathogens (bacteria, fungi, parasites, etc.) has led to significantly higher mortality rates from infectious diseases and currently presents a serious crisis worldwide. Despite the introduction of second and third generation antimicrobial drugs, certain pathogens, such as vancomycin resistant strains of Enterococcus faciearn, have developed resistance to all currently available drugs. New antimicrobial drugs must be discovered to treat infections by such organisms, and new methods are urgently needed to facilitate making such discoveries.

Neither whole cell screening, chemistry, nor target based drug discovery approaches as currently applied, have met the challenge of controlling infectious diseases, particularly those caused by drug resistant microorganisms. Technical advances in molecular biology, automated methods for high throughput screening, and chemical syntheses have led to an increase in the number of target based screens utilized for antimicrobial drug discovery and in the number of compounds being analyzed. However, despite these advances, only a limited number of antimicrobial drugs acting by a novel mechanism have been identified during recent years.

A number of potentially novel, valuable targets are incompatible with current methods to screen for drug candidates because either the target's exact function and molecular mechanism of action are unknown, or there are technical obstacles preventing the development of effective high throughput screening methods. It can take anywhere from six months to several years to develop a screening assay, which is impractical when the goal is to rapidly screen multiple targets in a cost-effective manner.

The path in the progression from target identification through assay development, high throughput screening, medicinal chemistry, lead optimization, preclinical and clinical drug development is expensive, time consuming and full of technical challenges. Many different targets must be screened against multiple chemical compounds to identify new lead compounds for drug development. New, efficient technologies are needed that can be broadly applied to a variety of different targets to validate targets in the direct context of the desired outcome of drug therapy and to rapidly develop screening assays using these targets for drug discovery. Such developments will allow the wealth of genomics information to be leveraged for drug discovery and will lower the risk and costs while expediting the timelines of the drug discovery process.

For example, nearly 40% of the Haemophilus influenzae genome is comprised of genes of unknown function, many of which have no recognizable functional orthologues in other species. Similar numbers of unidentified open reading frames (orfs) are present in other sequenced or partially sequenced genomes of infectious organisms. Comprehensive screens and selections for identifying functional classes of genes provide a crucial starting point for converting the vast body of growing sequence data into meaningful biological information that can be used for drug discovery.

One major and important class of genes are those bacterial genes that are essential for growth or viability of a bacterium. Because useful conventional antibiotics are known to act by interfering with the products of essential genes, it is likely that the discovery of new essential gene products will have a significant impact on efforts to develop novel antimicrobial drugs. Essential gene products have been traditionally identified through the isolation of conditional lethal mutants, or by transposon mutagenesis in the presence of a complementing wild type allele (balanced lethality). However, such approaches are laborious, as they require identification, purification, and study of individual mutant strains.

In part, in order to facilitate the discovery of novel anti-microbial drugs, it would be desirable to have a rapid, generalized method of identifying essential growth/viability genes in pathogens or other organisms. Such a method would be particularly useful for identifying essential genes in pathogens that are not genetically well-characterized. Such a method could also be used to identify essential genes in higher organisms, e.g., in animals and in plants.

Knowledge of genes or gene products essential to the growth of an organism can provide a key to the development of treatments of infectious pathogens.

### SUMMARY

Homologous recombination can be used to disrupt genes in a host cell by inserting an exogenous nucleotide sequence, such as a selectable marker, into a target gene. However, one problem in disrupting a gene simply by replacing it with an exogenous sequence is that the disruption may affect the expression of genes that are located downstream of the disrupted gene. These potential downstream effects complicate analysis of the essentiality or other function of a target gene because the disrupted target gene may not be the only gene product affected by the disruption. Such downstream effects may also complicate interpretation of experiments using anti-sense oligonucleotide to disrupt gene expression.

Accordingly, this invention provides methods which allow disruption of a desired target gene without creating any downstream effects (FIG. 1). In one exemplary embodiment, this is done using a targeting polynucleotide comprising a molecular tag (or bar-code) and flanking homology clamps for in-frame disruption of a target gene. In various embodiments, the molecular tag may be a random sequence that does not occur in the host cell or it may be a sequence encoding for a protein capable of generating a selectable or detectable signal (e.g., fluorescence, antibiotic resistance, etc.).

By this invention, the generation of an in-frame gene disruption makes it possible to alter the expression of the disrupted gene product without affecting the expression of downstream gene products. This result contrasts to the situation in which an alteration is made out-of-frame because any change in the phenotype of the host cell may be attributable to a change in the expression of one or more gene products downstream from the target gene.

In exemplary embodiments, the molecular tag may be inserted under the control of the transcriptional and/or translation control of the target gene (e.g., the start codon, termination sequences, etc. of the targeted gene).

In various embodiments, after transformation of the targeting polynucleotide into a host cell, the culture may be grown so that cells with a disrupted essential gene are diluted out. DNA from the amplified cells may then be extracted and analyzed, for example by PCR or hybridization to a nucleic acid array, to determine whether the culture contains cells with the inserted molecular tag. In one embodiment, amplification uses a first primer complementary to the molecular tag and a second primer complementary to a genomic region outside of the targeted region.

The methods and compositions disclosed herein may be used for the identification and/or characterization of essential, conditionally essential, and non-essential genes. In one embodiment, the methods disclosed herein may be used to identify an essential gene. In an exemplary embodiment, substantially all essential genes in a genome may be identified.

In certain embodiments, the methods and compositions disclosed herein are applicable to any organism (i) for which part or all of the genome sequence of the organism is known and (ii) is capable of carrying out, or may be engineered so as to carry out, homologous recombination. In an exemplary embodiment, homologous recombination is carried out using the phage λ recombinase system.

The methods of this application are applicable to both prokaryotic and eukaryotic organisms. For mammalian cells, the method may feature, for example, 1) generation of a ES cell line expressing a functional homologous recombination system (e.g., a lambda recombination system), 2) the generation of an initial knock-out cell line using high frequency homologous recombination (e.g., lambda enzyme-mediated recombination) with a selectable marker (e.g., Neomycin phosphotransferase, etc.), 3) a targeting polynucleotide which may be homologously recombined with the target gene to knock-out the second copy of the gene of interest (e.g., lambda enzyme-mediated recombination), 4) passage of cells to allow cells with a disrupted second copy of the essential gene to be diluted during growth, and 5) analysis of the passed cells for the presence of the molecular tag insert (e.g., using PCR or microarray analysis).

The above and further features and advantages of the invention are described in the following Detailed Description and claims. The claims appended hereto are hereby incorporated into the specification in their entirety.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press:1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., New York); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986). Transgenic mice are derived according to Hogan, et al., “Manipulating the Mouse Embryo: A Laboratory Manual”, Cold Spring Harbor Laboratory (1988) which is incorporated herein by reference.

Embryonic stem cells may be manipulated according to published procedures (Teratocarcinomas and embryonic stem cells: a practical approach, E. J. Robertson, ed., IRL Press, Washington, D.C., 1987; Zjilstra et al., Nature 342:435-438 (1989); and Schwartzberg et al., Science 246:799-803 (1989), each of which is incorporated herein by reference).

Zygotes may be manipulated according to known procedures; for example see U.S. Pat. No. 4,873,191, Brinster et al., PNAS 86:7007 (1989); Susulic et al., J. Biol. Chem. 49:29483 (1995), and Cavard et al., Nucleic Acids Res. 16:2099 (1988), hereby incorporated by reference.

Oligonucleotides, modified oligonucleotides and peptide nucleic acids may be made as is generally known in the art. For example, oligonucleotides may be synthesized on an Applied Bio Systems oligonucleotide synthesizer according to specifications provided by the manufacturer.

A number of methods are available for creating microarrays of biological samples, such as arrays of DNA samples to be used in DNA hybridization assays. Exemplary are PCT Application Serial No. WO95/35505, published Dec. 28, 1995; U.S. Pat. No. 5,445,934, issued Aug. 29, 1995; and Drmanac et al., Science 260:1649-1652. Yershov et al. (1996) Genetics 93:4913-4918 describe an alternative construction of an oligonucleotide array. The construction and use of oligonucleotide arrays is reviewed by Ramsay (1998).

Methods of using oligonucleotide arrays will be within the knowledge of one of ordinary skill in the art based on the teachings herein. For example, Milosavljevic et al. (1996) Genomics 37:77-86 describe DNA sequence recognition by hybridization to short oligomers. The use of arrays for identification of unknown mutations is proposed by Ginot (1997) Human Mutation 10:1-10.

Detection of known mutations is described in Hacia et al. (1996) Nat. Genet. 14:441-447; Cronin et al. (1996) Human Mut.7:244-255; and others. The use of arrays in genetic mapping is discussed in Chee et al. (1996) Science 274:610-613; Sapolsky and Lishutz (1996) Genomics 33:445-456; etc. Shoemaker et al. (1996) Nat. Genet. 14:450-456 perform quantitative phenotypic analysis of yeast deletion mutants using a parallel bar-coding strategy.

Quantitative monitoring of gene expression patterns with a complementary DNA microarray is described in Schena et al. (1995) Science 270:467. DeRisi et al. (1997) Science 270:680-686 explore gene expression on a genomic scale. Wodicka et al. (1997) Nat. Biotech. 15:1-15 perform genome wide expression monitoring in S. cerevisiae.

### BRIEF DESCRIPTION OF THE DRAWINGS

FIG. 1 is a schematic representation of a method for determining gene essentiality using targeted disruption without producing any downstream polar effects.

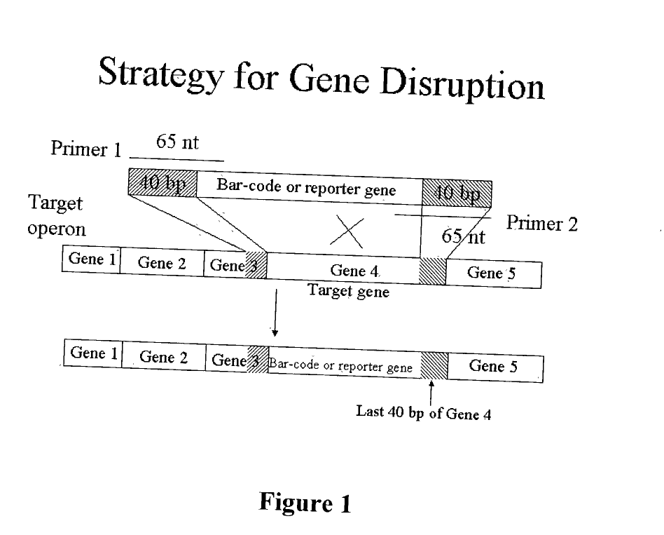

### DETAILED DESCRIPTION

To provide an overall understanding, certain illustrative embodiments will now be described; however, it will be understood by one of ordinary skill in the art that the systems and methods described herein can be adapted and modified to provide systems and methods for other suitable applications and that other additions and modifications can be made without departing from the scope of the systems and methods described herein.

Unless otherwise specified, the illustrated embodiments can be understood as providing exemplary features of varying detail of certain embodiments, and therefore unless otherwise specified, features, components, modules, and/or aspects of the illustrations can be combined, separated, interchanged, and/or rearranged without departing from the disclosed systems or methods.

1. Definitions

For convenience, certain terms employed in the specification, examples, and appended claims are collected here. Unless defined otherwise, all technical and scientific terms used herein have the same meaning as commonly understood by one of ordinary skill in the art to which this invention belongs. These terms should be understood and read in light of the specification as a whole.

The articles “a” and “an” are used herein to refer to one or to more than one (i.e., to at least one) of the grammatical object of the article. By way of example, “an element” means one element or more than one element.

The terms “cells” and “host cells” are used interchangeably herein and refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “complementary,” refers to nucleotide sequences on two nucleic acid molecules (e.g., two DNA molecules, two RNA molecules, or a DNA and an RNA molecule) that are capable of base pairing with each other (e.g., A:T and G:C nucleotide pairs). The complementarity between the two nucleic acid molecules, or regions thereof, need not be perfect and may, in various embodiments, be at least about 70%, 80%, 85%, 90%, 95, 96%, 97%, 98%, 99% or more complementary. For illustration, the nucleotide sequence “TATAC” corresponds to a reference sequence “TATAC” and is perfectly complementary to a reference sequence “GTATA”.

The terms “comprise” and “comprising” are used in the inclusive, open sense, meaning that additional elements may be included.

The terms “compound”, “test compound” and “molecule” are used herein interchangeably and are meant to include, but are not limited to, peptides, nucleic acids, carbohydrates, lipids, organic molecules, natural product extract libraries, and any other molecules, including, but not limited to, chemicals, metals, organometallic compounds, and inorganic compounds.

The term “detectable marker” refers to a polynucleotide sequence that facilitates the identification of a cell harboring the polynucleotide sequence. In certain embodiments, the detectable marker encodes for a chemiluminescent or fluorescent protein, such as, for example, green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED).

The term “downstream effects,” with reference to a disrupted target polynucleotide sequence, refers to any alteration in the rate and/or level of production of a gene product (e.g., RNA and/or protein) from a non-target polynucleotide sequence that is 3′ to the target polynucleotide sequence. By way of explanation, regions of a polynucleotide sequence may be described with reference to the position and direction (5′ to 3′) of transcription by RNA polymerase or translation by the ribosome. Downstream (or 3′ to) is in the direction of transcription (or translation) whereas upstream (5′ to) is in the direction from which the polymerase (or ribosome) has come. In certain instances, a target polynucleotide sequence and a downstream gene sequence rely on the same promoters and other regulatory sequences, so that, absent any alteration of the sequence of the genome, the gene product would have been transcribed (or translated) by a polymerase (or ribosome) that usually would have first transcribed (or translated) the target polynucleotide sequence (e.g., an operon encoding a polycistronic mRNA from a group of adjacent genes). Downstream effects do not refer to effects caused by a modification in the activity or level of expression of a gene product encoded by the target polynucleotide. Also, in certain embodiments, downstream effects do not refer to loss of a regulatory sequence (e.g., a promoter) that, absent any disruption in the genome, usually regulates a downstream gene but does not regulate the target polynucleotide sequence.

The term “essential gene” refers to a nucleic acid that encodes a polypeptide or RNA whose function is required for survival, growth, and/or mitosis/meiosis of a cell. Disruption of an essential gene may be lethal, i.e., prevent a cell from surviving, growing, or undergoing mitosis/meiosis. Alternatively, disruption of an essential gene may allow survival of a cell but result in severely diminished growth or metabolic rate. In certain embodiments, a gene may be essential under normal growth conditions. Alternatively, a gene may be “conditionally essential” indicating that the gene is essential under certain environmental conditions but not others. Conditionally essential genes may be essential under environmental conditions such as, for example, oxygen tension, osmolarity, pH, temperature, nutrient availability, phase of growth, presence of a test compound, and conditions encountered in a disease state, or combinations thereof. In certain embodiments, environmental conditions may refer to those encountered by a cell in vivo (e.g., in an organism or tissue) or in vitro (e.g., cells in tissue culture). Conditions encountered in a disease state may refer to conditions encountered in a diseased organism or to simulated disease conditions (e.g, an animal model or tissue culture model of disease). In an exemplary embodiment, a disease state refers to an infection by a biological pathogen.

The term “gene” refers to a nucleic acid comprising an open reading frame encoding a polypeptide having exon sequences and optionally intron sequences. The term “intron” refers to a DNA sequence present in a given gene which is not translated into protein and is generally found between exons.

The term “homologous recombination” refers to a process by which an exogenously introduced DNA molecule integrates into a target DNA molecule in a region where there is identical or near-identical nucleotide sequence between the two molecules. Homologous recombination is mediated by complementary base-pairing, and may result in either insertion of the exogenous DNA into the target DNA (a single cross-over event), or replacement of the target DNA by the exogenous DNA (a double cross-over event). Homologous recombination may occur in any cell having a homologous recombination system. A “homologous recombination system” refers to one or more polypeptides that facilitate homologous recombination in a cell. Homologous recombination systems may be endogenous to the cell or may be introduced into the cell using recombinant technology. In an exemplary embodiment, a homologous recombination system refers to one or more of the exo, bet (β) and gam (γ) genes from phage lambda (λ). Homologous recombination may occur in virtually any cell type, including bacterial, mycobacterial, yeast, fungal, algal, plant, or animal (including mammalian and isolated human) cells.

The term “identity” refers to the percentage of identical nucleotide residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

The term “including” is used herein to mean “including but not limited to”. “Including” and “including but not limited to” are used interchangeably.

The term “in-frame,” with reference to a nucleotide sequence, refers to the translational reading frame of the sequence. The translational reading frame of a sequence may be determined by reading nucleotides in groups of three from an initiation codon. A mutation of the nucleotide sequence which inserts or deletes one or more nucleotides but not a multiple of three may lead to a change in the translational reading frame (i.e., a frameshift) resulting in a change in the encoded sequence occurring at the site of mutation and downstream therefrom.

An “in-frame disruption” refers to an alteration of a target nucleotide sequence (e.g., insertion, deletion, or other alteration of the sequence) that is made in frame and thereby does not lead to a frameshift, i.e., maintains the translational reading frame of the target sequence and any downstream sequences. In certain instances, an in-frame disruption may alter the entire sequence of a gene product, so that the determination as to whether the alteration was made in-frame is made by reference to downstream nucleotide sequences and gene products encoded thereby. By way of contrast, an alteration to a target nucleotide sequence that is not in-frame, and therefore does not give rise to an in-frame disruption, would not maintain the translational reading frame of the target sequence or a sequence located downstream of the target sequence.

The term “isolated” as used herein with respect to nucleic acids, such as DNA or RNA, refers to molecules separated from other DNAs, or RNAs, respectively, that are present in the natural source of the macromolecule. For example, isolated nucleic acids encoding a polypeptide preferably include no more than 10 kilobases (kb) of nucleic acid sequence which naturally immediately flanks a particular gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state.

The terms “nucleic acid”, “oligonucleotide”, and “polynucleotide,” or grammatical equivalents herein, refer to at least two nucleotides covalently linked together. A nucleic acid of the present invention will generally contain phosphodiester bonds, although in some cases nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate, phosphorodithioate, O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). These modifications of the ribose-phosphate backbone or bases may be done to facilitate the addition of other moieties such as chemical constituents, including 2′O-methyl and 5′ modified substituents, or to increase the stability and half-life of such molecules in physiological environments. In various embodiments, nucleic acids may be single stranded or double stranded, or may contain portions of both double stranded or single stranded sequence. Nucleic acids may be DNA, either genomic or cDNA, RNA, or a hybrid, where the nucleic acid contains any combination of deoxyribo-and ribonucleotides, and any combination of bases, including uracil, adenine, thymine, cytosine, guanine, inosine, xathanine and hypoxathanine, etc. Thus, for example, chimeric DNA-RNA molecules may be used in accordance with certain embodiments of the methods described herein (see e.g., Cole-Strauss et al., Science 273:1386 (1996) and Yoon et al., PNAS USA 93:2071 (1996), both of which are hereby incorporated by reference).

The term “operably linked”, when describing the relationship between two nucleic acid regions, refers to a juxtaposition wherein the regions are in a relationship permitting them to function in their intended manner. For example, a control sequence (e.g., a transcriptional regulatory sequence, etc.) “operably linked” to a coding sequence is ligated in such a way that expression of the coding sequence is achieved under conditions compatible with the control sequences, such as when the appropriate molecules (e.g., inducers and polymerases) are bound to the control or regulatory sequence(s).

The term “or” as used herein should be understood to mean “and/or”, unless the context clearly indicates otherwise.

The term “pathogen” refers to any organism which is capable of infecting an animal or plant and replicating its nucleic acid sequences in the cells or tissue of the animal or plant. Such a pathogen is generally associated with a disease condition in the infected animal or plant. Such pathogens may include, but are not limited to, viruses, which replicate intra- or extracellularly, or other organisms such as bacteria, fungi or parasites, which generally infect tissues or the blood. Certain pathogens are known to exist in sequential and distinguishable stages of development, e.g., latent stages, infective stages, and stages which cause symptomatic diseases. In these different states, the pathogen is anticipated to rely upon different genes as essential for survival. In exemplary embodiments, pathogens include, for example, Streptococcus pneumoniae, Staphylococcus aureus, Enterococcus faecalis, Pseudomonas aeruginosa, Helicobacter pylori and Escherichia coli.

The term “phenotype” refers to the entire physical, biochemical, and physiological makeup of a cell, e.g., having any one trait or any group of traits.

The terms “recombinant protein” or “recombinant polypeptide” refer to a polypeptide encoded by a polynucleotide produced by recombinant DNA techniques. An example of such techniques includes the case when DNA encoding the expressed protein is inserted into a suitable expression vector which is in turn used to transform a host cell to produce the protein or polypeptide encoded by the DNA. A host cell containing a polynucleotide produced by recombinant DNA techniques is referred to as a “recombinant cell.” In certain embodiments, the polynucleotide may be inserted into the genome of the recombinant cell or may be maintained extrachromosomally.

The term “reporter gene” refers to any gene which encodes a product whose expression is detectable and/or quantitatable by immunological, chemical, biochemical, biological, mechanical, or other types of assays. A reporter gene product may, for example, have one of the following attributes, without restriction: fluorescence (e.g., green fluorescent protein), enzymatic activity (e.g., lacZ/β-galactosidase, luciferase, chloramphenicol acetyltransferase, alkaline phosphatase, etc.), toxicity (e.g., ricin), or an ability to be specifically bound by a second molecule (e.g., biotin or a detectably labeled antibody). It is understood that any engineered variants of reporter genes, which are readily available to one skilled in the art, are also included, without restriction, in the foregoing definition.

The term “reporter gene construct” refers to a nucleic acid that includes a “reporter gene” operatively linked to a transcriptional regulatory sequence. Transcription of the reporter gene is controlled at least in part by these sequences. The transcriptional regulatory sequences can include a promoter and other regulatory regions, such as enhancer sequences, that modulate the level of expression of a reporter gene in response to the level and/or activity of another protein.

The term “selectable marker” refers to a polynucleotide sequence encoding a gene product that alters the ability of a cell harboring the polynucleotide sequence to grow or survive in a given growth environment relative to a similar cell lacking the selectable marker. Such a marker may be a positive or negative selectable marker. For example, a positive selectable marker (e.g., an antibiotic resistance or auxotrophic growth gene) encodes a product that confers growth or survival abilities in selective medium (e.g., containing an antibiotic or lacking an essential nutrient). A negative selectable marker, in contrast, prevents polynucleotide-harboring cells from growing in negative selection medium, when compared to cells not harboring the polynucleotide. A selectable marker may confer both positive and negative selectability, depending upon the medium used to grow the cell. The use of selectable markers in prokaryotic and eukaryotic cells is well known by those of skill in the art. Suitable positive selection markers include, e.g., neomycin, kanamycin, hyg, hisD, gpt, bleomycin, tetracycline, hprt SacB, beta-lactamase, ura3, ampicillin, carbenicillin, chloramphenicol, streptamycin, gentamycin, phleomycin, and nalidixic acid. Suitable negative selection markers include, e.g., hsv-tk, hprt, gpt, and cytosine deaminase.

The terms “signal transduction,” “signaling,” “signal transduction pathway,” “signaling pathway,” etc. are used herein interchangeably and refer to the processing of physical or chemical signals from the extracellular environment through the cell membrane, and may occur through one or more of several mechanisms, such as activation/inactivation of enzymes (such as proteases, or other enzymes which may alter phosphorylation patterns or other post-translational modifications), activation of ion channels or intracellular ion stores, effector enzyme activation via guanine nucleotide binding protein intermediates, formation of inositol phosphate, activation or inactivation of adenylyl cyclase, direct activation (or inhibition) of a transcriptional factor, etc.

The term “small molecule” refers to a composition that has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures comprising arrays of small molecules, often fungal, bacterial, or algal extracts, which can be screened with any of the assays of the invention.

The term “specifically hybridizes” refers to the ability of a nucleic acid to hybridize to at least 15, 25, 50, 100, or more, consecutive nucleotides of a target gene sequence, or a sequence complementary thereto, or naturally occurring mutants thereof, such that it has less than 15%, preferably less than 10%, and more preferably less than 5% background hybridization to a cellular nucleic acid (e.g., mRNA or genomic DNA) other than the target gene.

The term “suitable for PCR,” when used in reference to a sequence of DNA, indicates that PCR may be performed on such sequence of DNA because the necessary PCR primers may be identified.

The terms “target gene,” “target sequence,” or “targeted sequence” refer to a nucleotide sequence that may be disrupted by homologous recombination with a targeting polynucleotide. In an exemplary embodiment, a target gene is an open reading frame found within a genome that encodes a polypeptide. Disruption of a target gene with a targeting polynucleotide in the manner of the present invention creates a “disrupted gene.”

The term “targeting polynucleotide” refers to a nucleotide sequence that may be inserted at a desired location in a target sequence through homologous recombination. In exemplary embodiments, the targeting polynucleotide comprises a molecular tag flanked by homology clamps. A “molecular tag” (also referred to as a bar code) refers to a nucleotide sequence contained in the targeting polynucleotide that may be used to detect, identify, characterize and/or isolate a targeting polynucleotide, or a target sequence into which a targeting polynucleotide has been introduced via homologous recombination. In exemplary embodiments, the molecular tag may be a random sequence which does not naturally occur in the organism to which it is being introduced or it may be a sequence encoding for a selectable or detectable marker (i.e., a reporter gene). The molecular tag is usually flanked on one or both sides by “homology clamps” which guide the tag to a desired location within the genome by nucleotide complementarity, often a target gene. In exemplary embodiments, the homology clamps are nucleotide sequences which are at least about 10 nucleotides long, at least about 30 nucleotides long, at least about 36 nucleotides long, at least about 40 nucleotides long, at least about 45 nucleotides long, or at least about 50 to 100 nucleotides long. The targeting polynucleotide may additionally comprise any polynucleotide sequences that are desired to be introduced into a target sequence (e.g., by insertion into or replacement of the target sequence), any polynucleotide sequences that may facilitate homologous recombination at a target sequence, or any modifications that may increase its stability and/or enhance its uptake by a host cell.

The term “test compound” refers to any compound which is potentially capable of associating with a protein, and/or inhibiting or enhancing its enzymatic activity or its ability to interact with another molecule. The test compound may be designed or obtained from a library of compounds which may comprise peptides, as well as other compounds, such as small organic molecules and particularly new lead compounds. By way of example, the test compound may be a natural substance, a biological macromolecule, or an extract made from biological materials such as bacteria, fungi, or animal (particularly mammalian) cells or tissues, an organic or an inorganic molecule, a synthetic test compound, a semi-synthetic test compound, a carbohydrate, a monosaccharide, an oligosaccharide or polysaccharide, a glycolipid, a glycopeptide, a saponin, a heterocyclic compound, a structural or functional mimetic, a peptide, a peptidomimetic, a derivatized test compound, a peptide cleaved from a whole protein, or a peptides synthesized synthetically (such as, by way of example, either using a peptide synthesizer or by recombinant techniques or combinations thereof), a recombinant test compound, a natural or a non-natural test compound, a fusion protein or equivalents thereof and mutants, derivatives or combinations thereof.

The term “transcriptional regulatory sequence” refers to DNA sequences, such as initiation signals, enhancers, and promoters, which induce or control transcription of protein coding sequences with which they are operably linked. It will be understood that a gene can be under the control of transcriptional regulatory sequences which are the same or which are different from those sequences which control transcription of the naturally-occurring form of the gene.

The term “transformation” refers to any method for introducing foreign molecules, such as DNA, into a cell. Lipofection, DEAE-dextran-mediated transfection, microinjection, protoplast fusion, calcium phosphate precipitation, retroviral delivery, electroporation, natural transformation, and biolistic transformation are just a few of the methods known to those skilled in the art which may be used.

The term “vector” refers to a nucleic acid molecule capable of transporting another nucleic acid to which it has been linked. One type of vector which may be used in accord with the application is an episome, i.e., a nucleic acid capable of extra-chromosomal replication. Other vectors include those capable of autonomous replication and expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA molecules which, in their vector form are not bound to the chromosome. In the present specification, “plasmid” and “vector” are used interchangeably as the plasmid is the most commonly used form of vector. However, the application is intended to include such other forms of expression vectors which serve equivalent functions and which become known in the art subsequently hereto.

2. Targeting Polynucleotides

Targeting polynucleotides are nucleic acid constructs comprising a molecular tag and one or more homology clamps that may be used in accordance with the methods described herein to disrupt a target polynucleotide sequence by homologous recombination. In the various embodiments of the invention, the targeting polynucleotide is inserted into a target sequence in-frame without causing downstream effects on sequences located 3′ to the target sequence. In various embodiments, disruption of the target sequence may involve insertion of part or all of the targeting polynucleotide into the target sequence or replacement of all or part of a target sequence with sequences from the targeting polynucleotide.

In other embodiments, targeting polynucleotides may additionally comprise any polynucleotide sequences that are desired to be introduced into a target sequence (e.g., by insertion into or replacement of the target sequence or a portion thereof), any polynucleotide sequences that may facilitate homologous recombination at a target sequence, or any modifications that may increase the stability and/or enhance the uptake of the targeting polynucleotide by a host cell. Exemplary sequences which may be useful in accordance with the targeting polynucleotides and methods described herein, include, for example, a nucleotide sequence encoding a polypeptide, or derivates or fragments thereof, which are desired to be expressed in a host cell.

In one embodiment, a targeting polynucleotide may comprise a reporter gene encoding a selectable or detectable marker. In an exemplary embodiment, a selectable or detectable marker contained on a targeting polynucleotide may be inserted under the control of the transcriptional and/or translational regulatory sequences of a target gene and may be used as an initial screen to determine that the targeting polynucleotide was inserted into the genome of the host cell in-frame with respect to the initiation codon of the disrupted gene.

In another embodiment, a targeting polynucleotide may comprise a modified version of a sequence that is endogenous to the host cell. In such cases, the modified sequence can replace the endogenous sequence by homologous recombination thus creating a mutant version of the sequence in the host cell (e.g., one or more polynucleotide substitutions, deletions or insertions).

In still other embodiments, a targeting polynucleotide may comprise a polynucleotide sequence capable of modulating transcriptional regulation of a gene. In various embodiments, the transcriptional regulatory sequences may be operably linked to sequences found on the targeting polynucleotide or may be inserted into the target sequence so as to be operably linked to an endogenous sequence in the host cell.

In-frame disruption of a target gene by a targeting polynucleotide may be confirmed by a variety of methods. In an exemplary embodiment, in-frame disruption may be confirmed by PCR using a primer directed to a region of the targeting polynucleotide that is inserted into the host genome and a primer directed to a region of the host genome located outside of the target sequence. Such a primer pair will amplify a region containing a junction between the genomic sequence and the inserted sequence. Proper insertion may be determined based on the size of the amplified fragment, by determining the sequence of the amplified fragment, or by hybridizing the amplified fragment to a microarray.

In certain embodiments, the targeting polynucleotide may be directed to a desired gene in order to determine its essentiality to the organism. In other embodiments, multiple genes, e.g., at least 2, 10, 50, 100 or 1,000 genes, may be targeted for disruption by homologous recombination. In still other embodiments, substantially all the genes in the genome of an organism may be targeted for systematic disruption by homologous recombination in order to determine the essentiality of each gene in the genome. When more than one gene is being targeted for disruption, it is possible to carry out the methods of the invention in parallel, e.g., different targeting polynucleotides are introduced into separate cultures, or in batch, e.g., different targeting polynucleotides are introduced into a single culture. In certain embodiments, all genes may be disrupted with a common molecular tag flanked by different homology clamps specific for the desired target gene. In alternative embodiments, each gene may be disrupted with a unique molecular tag flanked by different homology clamps specific for the desired target gene. When each gene is being disrupted with a unique molecular tag, it may be desirable for the targeting polynucleotide to further comprise common priming sites so as to allow amplification of the targeted region for the entire population in a common reaction using common PCR primers.

In one embodiment, in-frame disruption with a targeting polynucleotide may be used to characterize the essentiality of a gene in the absence of any downstream effects. In an exemplary embodiment, identification of an essential gene may be carried out by performing PCR footprinting on a pool of cells transformed with a targeting polynucleotide. The PCR footprinting is performed using a primer that hybridizes to the targeting polynucleotide, plus a primer that hybridizes to a specific location on the chromosome, after which the PCR products are separated on a footprinting gel. A PCR product on the gel represents a region of the chromosome that does not contain an essential gene, and the lack of a PCR product in an area of the gel, where a PCR product is expected, represents a region of the chromosome that contains an essential gene. Alternatively, a low level of the PCR product on the gel, relative to other PCR products on the gel, represents a region of the chromosome that contains an essential gene. In another embodiment, the identification of an essential region of DNA is analyzed by hybridizing the amplified tags to an oligonucleotide array containing tag sequences in the population. In certain embodiments, it may be desirable to amplify the DNA using PCR before hybridization of the DNA to the array.

In another embodiment, where the targeting polynucleotide contains a reporter gene, identification cells that successfully inserted the targeting polynucleotide into the correct location in-frame may be based on a reporter gene assay, wherein a cell having the polynucleotide insertion expresses the reporter gene and a cell lacking the polynucleotide insertion does not express the reporter gene. In one embodiment, the reporter gene may be a positive or negative selectable marker. Exemplary positive selectable markers include, for example, neomycin, kanamycin, hyg, hisD, gpt, bleomycin, tetracycline, hprt SacB, beta-lactamase, ura3, ampicillin, carbenicillin, chloramphenicol, streptamycin, gentamycin, phleomycin, and nalidixic acid. Exemplary negative selectable markers, include, for example, hsv-tk, hprt, gpt, and cytosine deaminase. In an exemplary embodiment, a positive selectable marker may be used and cells containing disrupted DNA may be identified based upon the ability of the cells to grow on selective medium, wherein a cell containing a targeting polynucleotide can grow on selective medium, and a cell lacking a targeting polynucleotide cannot grow, or grows more slowly, on selective medium. In other embodiments, the reporter gene may be a detectable marker, such as green fluorescent protein. If the detectable marker is inserted under the control of the endogenous gene, it may be used as an initial screen to determine that the targeting polynucleotide was inserted into the proper location within the genome. After passage of the cells, the detectable marker may be used to assay for gene essentiality, wherein absence of the detectable marker is indicative of disruption of an essential gene. In one embodiment, the reporter gene encodes for a chemiluminescent or fluorescent protein, such as, for example, green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED). In other embodiments, the selectable and/or detectable marker may be used to characterize the essentiality of a gene. For example, a culture of cells wherein an essential gene has been disrupted in-frame using the methods of the invention may be passed to permit cells with a decreased growth rate or viability to be diluted out of the culture. A change in the amount of cells expressing the selectable or detectable marker as compared to non-disrupted cells indicates that the disrupted gene had an effect on the growth rate or viability of the host cell.

Targeting polynucleotides may be produced using a variety of art recognized techniques, including, for example, chemical synthesis of oligonucleotides, nick-translation of a double-stranded DNA template, polymerase chain-reaction amplification of a sequence (or ligase chain reaction amplification), purification of prokaryotic or target cloning vectors harboring a sequence of interest (e.g., a cloned cDNA or genomic clone, or portion thereof) such as plasmids, phagemids, YACs, cosmids, bacteriophage DNA, other viral DNA or replication intermediates, or purified restriction fragments thereof, as well as other sources of single and double-stranded polynucleotides having a desired nucleotide sequence. Targeting polynucleotides are generally ssDNA or dsDNA.

Targeting polynucleotides are generally at least about 5, 10, 12, 15, 20, 25, 30, 40, 50, 80, 100, 150, 200, 250, 300, 400, 500, 800, 1000, 1500, or 2000 nucleotides in length, or longer, and comprise at least one homology clamp sequence that substantially corresponds to, or is substantially complementary to, a predetermined target nucleotide sequence. In various embodiments, a target nucleotide sequence may be an endogenous sequence (i.e., a DNA sequence of a polynucleotide located in a target cell, such as a chromosomal, mitochondrial, chloroplast, viral, episomal, or mycoplasmal polynucleotide) or an exogenous nucleotide sequence that has been introduced into a host cell. Such homology clamps serve as guides for homologous recombination with the predetermined target sequence.

In various embodiments, homology clamps may be located at or near the 5′ and/or 3′ end(s) of the targeting polynucleotide. In exemplary embodiments, homology clamps are located at or near each end of the targeting polynucleotide. Homology clamps typically form one or more subsequences of the targeting polynucleotide construct and must be of sufficient length to effectively direct the targeting polynucleotide to a desired target sequence within a cell. For example, such homology clamps may be at least about 2, 3, 4, 5, 6, 7, 8, 9, 10, 11, 12, 13, 14, 15, 20, 22, 25, 30, 35, 40, 50, or 100 nucleotides in length. The optimal and minimal clamp lengths may be determined by the skilled artisan based on the degree of sequence homology between the homology clamp and the target sequence and the base composition of the target sequence (e.g., G-C rich sequences are typically more thermodynamically stable and will generally require shorter clamp length). Therefore, both homology clamp length and the degree of sequence homology may be determined with reference to a particular predetermined sequence. In exemplary embodiments, homology clamps are at least about 12 nucleotides long and substantially correspond, or are substantially complementary to, a predetermined target sequence. In one embodiment, a homology clamp is 50 nucleotides long and is identical to or complementary to a predetermined target sequence.

In certain embodiments, the targeting polynucleotides, described herein may be derivatized with one or more chemical substituents that modulate the function of the targeting polynucleotide or cause an alteration or chemical modification to a nucleotide sequence at or near the target sequence. Examples of chemical substituents include, but are not limited to, cross-linking agents, nucleic acid cleavage agents, metal chelates (e.g., iron/EDTA chelate for iron catalyzed cleavage), topoisomerases, endonucleases, exonucleases, ligases, phosphodiesterases, photodynamic porphyrins, chemotherapeutic drugs (e.g., adriamycin, doxirubicin), intercalating agents, labels (including, for example, fluorescent or chemiluminescent labels), base-modification agents, 2′-O methyl groups, immunoglobulin chains, and oligonucleotides. See for example Kutryavin et al., Biochem. 35:11170 (1996); Woo et al., Nucleic Acid. Res. 24(13):2470 (1996); Podyminogin et al., Biochem. 34:13098 (1995) and 35:7267 (1996); Hertzberg et al. (1982) J. Am. Chem. Soc. 104: 313; Hertzberg and Dervan (1984) Biochemistry 23: 3934; Taylor et al. (1984) Tetrahedron 40: 457; Dervan, P B (1986) Science 232: 464); Afonina et al., PNAS USA 93:3199 (1996); Cole-Strauss et al., Science 273:1386 (1996); and Yoon et al., PNAS 93:2071 (1996); which are incorporated herein by reference. In various embodiments, attachment chemistries for derivatizing a targeting polynucleotide with a chemical substituent include direct linkage via a reactive amino group (Corey and Schultz (1988) Science 238:1401, which is incorporated herein by reference), streptavidin/biotin, digoxigenin/antidigoxigenin antibody, and other linkage methods. See e.g., U.S. Pat. Nos. 5,135,720, 5,093,245, and 5,055,556, which are incorporated herein by reference.

In other embodiments, a targeting polynucleotide may be conjugated, by covalent or noncovalent binding, to a cell-uptake component. Suitable cell-uptake components include various polypeptides, lipids, or combinations thereof that are known in the art, including, for example, lipid vesicles made according to Feigner (WO91/17424, incorporated herein by reference), cationic lipidization (WO91/16024 and EP 465,529, incorporated herein by reference) and/or nucleases. In certain embodiments, a cell-uptake component may target a polynucleotide to a specific cell type. For example, a targeting polynucleotide can be conjugated to an asialoorosomucoid (ASOR)-poly-L-lysine conjugate for targeting to hepatocytes (Wu G Y and Wu C H (1987) J. Biol. Chem. 262:4429; Wu G Y and Wu C H (1988) Biochemistry 27:887; Wu G Y and Wu C H (1988) J. Biol. Chem. 263:14621;Wu G Y and Wu C H (1992) J. Biol. Chem. 267: 12436; Wu et al. (1991) J. Biol. Chem. 266: 14338; and Wilson et al. (1992) J. Biol. Chem. 267: 963, WO92/06180; WO92/05250; and WO91/17761, which are incorporated herein by reference). In other embodiments, a targeting polynucleotide may comprise other components such as a nuclear localization signal for facilitating entry of the polynucleotide into the nucleus of a cell, as is known in the art.

In various embodiments, targeting polynucleotides may be isolated, linear polynucleotides or may be incorporated into a vector. Targeting polynucleotides may be introduced into a prokaryotic or eukaryotic host cell using well-known methods. For example, a variety of methods may be used to introduce a polynucleotide into a host cell, including, for example, microinjection, calcium phosphate treatment, electroporation, lipofection, biolistics or viral-based transfection. Other methods used to transform mammalian cells include the use of Polybrene, protoplast fusion, and others (see, generally, Sambrook et al. Molecular Cloning: A Laboratory Manual, 2d ed., 1989, Cold Spring Harbor Laboratory Press, Cold Spring Harbor, N.Y., which is incorporated herein by reference). Direct injection of DNA and/or recombinase-coated targeting polynucleotides into target cells, such as skeletal or muscle cells also may be used (Wolff et al. (1990) Science 247: 1465, which is incorporated herein by reference).

In certain embodiments, it may be desirable to introduced the targeting polynucleotide into a host cell simultaneously or contemporaneously with a recombinase protein to facilitate homologous recombination. In exemplary embodiments, the targeting polynucleotide is preincubated with recombinase so as to form a recombinase coated targeting polynucleotide wherein the recombinase is noncovalently bound to the polynucleotide. See e.g., See e.g., U.S. Pat. No. 6,200,812. In exemplary embodiments, the recombinase is the recA protein from E. coli.

3. Molecular Tags

The targeting polynucleotides used in accordance with compositions and methods disclosed herein may comprise a molecular tag. When disrupting multiple genes, the same molecular tag may be inserted into each gene or different molecular tags may be inserted into the different genes. In an exemplary embodiment, substantially all of the genes in a given genome may be systematically disrupted in-frame with targeting polynucleotides, wherein each gene is disrupted with a targeting polynucleotide comprising a unique molecular tag. Molecular tags may be from about 5-100 nucleotides in length, or about 5-50, or about 10-20, or about 10, 15, or 20 nucleotides in length. In exemplary embodiments, the molecular tags may be designed so that all tags in a population of strains having disrupted genes can be amplified with a single set of common primers (see, e.g., Shoemaker et al., 1996 Nature Genetics 14:450, which is incorporated herein by reference in its entirety) and quantitatively identified by hybridization. This embodiment thus facilitates analysis of a large number of strains, each of which contains a deletion or other target gene alteration associated with a unique tag, in a highly parallel fashion. Moreover, use of molecular tags allows pooling of strains having differing target gene expression levels (e.g., pooling of heterozygote and diploid strains).

Methods for preparation of molecular tags are well known in the art. For example, a selectable marker, such as a kanamycin resistance gene, may be amplified using a pair of long primers (e.g., from about 50-100 nucleotides in length). The first primer contains a targeting sequence that has homology to the 5′ end of the target sequence to be disrupted, while the second primer contains a targeting sequence having homology to the 3′ end of the target sequence. One of either the first or second primers is also designed to contain a molecular tag and, optionally, common priming sites flanking the molecular tag sequence for amplifying the tag sequence. Common priming sites refer to sequences for which PCR primers may be developed and are common to all molecular tags used in any one set of strains to be produced in accordance with the methods disclosed herein. Common tag priming sites may be, for example, from about 5-50 nucleotides in length, about 5-20, or about 15, 18 or 20 nucleotides in length. Thus, using PCR primers directed to the common tag priming sites, substantially all strains comprising a targeting polynucleotide insertion can be amplified using a single set of primer sequences. In an exemplary embodiment, an in-frame gene disruption is first analyzed to ensure that the insertion has occurred in-frame as described further herein.

In certain embodiments, a host cell used in accordance with the methods disclosed herein may be a diploid or a haploid cell. When a diploid strain is used, a heterozygous (e.g., altering one of the target sequences) or homozygous (e.g., altering both copies of the target sequence) disrupted strain can be constructed by introducing the amplified selectable marker into the host cell's genome in a site-specific manner. In an exemplary embodiment, the targeting polynucleotide may be used to create a heterozygous deletion strain by disrupting a target gene so as to substantially reduce expression of an RNA or protein product from the target gene. Where the host cell is a yeast strain, the amplified selectable marker containing the molecular tag can be transformed into a haploid yeast strain, the marker integrated into the target sequence by homologous recombination, and the resulting haploid deletion strain mated to produce a diploid strain that is heterozygous for the target sequence. More simply, the amplified selectable marker can be transformed into a diploid yeast strain to directly produce a heterozygous deletion strain (e.g., for essential genes). In exemplary embodiments, a strain may be constructed which is heterozygous for the target gene but is otherwise diploid for all other non-target sequences.

In another embodiment, the strains used in the invention contain two tags for each gene to be analyzed. Construction of strains containing two tags is similar to that described above for construction of strains containing a single tag, with the exception that both the primer containing homology to the 5′ sequence of the target gene and the primer containing homology to the 3′ region of the target gene contain tag priming sties and tag sequences. Use of two tags increases the sensitivity and multiplies the probability of identifying a strain correctly. Moreover, use of two tags decreases the incidence of errors or other problems in strain identification due to errors associated with tag synthesis or mutation of tag sequences during growth. Given the description of these two tagging strategies, it will be apparent to one of ordinary skill in the art that these strategies can be modified to construct strains having any number of tags, as well as construction of strains having tags that render one or more target sequences with which a tag is associated non-functional.

Strains disrupted in-frame with a targeting polynucleotide sequence may allow altered expression of a target sequence due to the presence of a heterologous promoter (e.g., an inducible promoter or a promoter having a promoter strength different from the native target gene promoter). Such strains can be constructed by introduction of the molecular tag sequence concomitant with replacement of the native promoter. It is not necessary that the molecular tag be at or near the site of the genomic alteration (e.g., the site of the site-specific deletion or at the site of promoter alteration). Rather, it is only necessary that the molecular tag be present within the same cell, preferably on a stable episomal element or in the genome, in a manner that does not affect any functional genes in the strain (i.e., does not affect strain growth or fitness) and uniquely identifies the strain (e.g., the molecular tag identifies the strain as the strain that contains the particular site-specific deletion, or the particular promoter alteration).

4. Characterization of Disrupted Genes

Host cells containing an in-frame target gene disrupted by homologous recombination with a targeting polynucleotide can be grown under any of a variety of conditions to characterize and/or identify gene products that are essential, conditionally essential, or non-essential to the growth rate and/or viability of the host cell. When using prokaryotes, effects of disruption of the single copy of the target sequence may be determined. When using eukaryotes, effects of disruption of one (heterozygous) or both (homozygous) copies of the target sequence may be examined. In an exemplary embodiment, the methods discussed herein may be used to identify one or more essential genes in an organism. In other embodiments, genes which are conditionally essential under certain environmental conditions may be identified. For example, genes may conditionally essential under one or more environmental conditions, such as various oxygen tensions, temperature extremes (e.g., high or low temperature), varying ionic conditions (e.g., high concentrations of salt), pH extremes (e.g., acid or basic culture conditions), nutrient availability, prolonged stationary phase, presence of a test compound, or under conditions encountered in a disease state, or combinations thereof. Of particular interest is the identification of gene products important in the growth of strains in the presence of a test compound. In exemplary embodiments, strains grown in the absence of a test compound may be used as a control. Strains can be grown in either liquid or on solid medium, preferably liquid medium. Where the strains are grown in liquid culture, the strains can be grown in small volumes (e.g., a volume of about 100 μl, about 200 μl, about 300 μl, about 500 μl, about 1 ml, or about 5 ml). In exemplary embodiments, the strains may be grown in microtiter plates such as 48-well, 96-well, or 384-well plates.

Differences in growth rate can be assessed by any of a variety of means well known in the art. For example, growth rate can be determined by measuring optical density (OD) as a function of time according to methods well known in the art. In general, as used herein, “growth rate” means the generation time or doubling time of the host cell. Thus an increase in growth rate is associated with an decrease in generation time or doubling time, while a decrease in growth rate is associated with an increase in generation time or doubling time. Growth rate differences as small as about 5% and even less than or about 1% can be detected using the methods described herein. In various embodiments, the growth rate of a single disrupted strain may be determined as compared to a non-disrupted strain, or the growth rates of two or more disrupted strains may be compared by examining the growth rates of the strains in parallel disrupted cultures or in a single culture containing a pool of disrupted strains. In an exemplary embodiment, the growth rate may be determined by competitively growing a pool of disrupted strains wherein the starting pool is composed of strains at equal abundance. Over time, strains which have a disrupted gene important for growth rate of the cell will be diluted in the culture. Identification of the presence and/or amount of the molecular tags associated with each of the disrupted genes may be determined using PCR amplification of the tag coupled with a detection means such as gel electrophoresis. Alternatively, the molecular tags may be analyzed using DNA microarray hybridization either directly on DNA fragments from the cells or using PCR amplified fragments. A change in the level of a molecular tag as compared to the starting culture will be indicative of a strain which has been disrupted in a gene important for growth rate of the cell.

In exemplary embodiments, the methods disclosed herein can be used to identify the gene product targets of a test compound that affects the growth rate and/or viability of a cell. Suitable test compounds may include, for example, antibiotics (e.g., antibacterial, bacteriostatic, and antifungal agents), chemotherapeutic agents, agents that affect (inhibit or enhance) a biosynthetic pathway, and the like.

In certain embodiments, a disrupted strain can be made even more sensitive to slight differences in expression or function of a target gene by globally decreasing transcription of all genes, e.g., by use of actinomycin in the cultures. The effects of target gene expression upon growth rate can also be examined under differing growth conditions, e.g., in media of differing nutrient composition (e.g., to simulate differing in vivo environments), as well as differing temperatures (e.g., to simulate the body temperature of a subject that may receive therapy using the test compound being examined).

After growth under a test condition as described above, the effect upon strain growth rate is analyzed. Where the strains are designed to contain a molecular tag, the relative abundance of each of the tagged strains can be determined by amplifying the tags using conventional PCR methods and an appropriate primer pair (either common primers for a pool of disrupted strains or a primer pair unique to an individual disrupted strain). The amplified tags are then analyzed to compare, quantitatively and/or qualitatively, the relative amounts of, for example, each molecular tag in a sample. The relative amounts of the tags are correlated to the relative abundance of the strains in the sample.

Analysis of the amplified tags can be accomplished according to any of a variety of methods well known in the art that allows for differentiation of the tag sequences. For example, where the tag sequences are of sufficiently different lengths, the tag composition in a sample of amplified tags can be analyzed using Southern hybridization techniques, or by hybridization to filters having bound sequences complementary to the tags.

In one embodiment, host cells which have been disrupted in-frame with a targeting polynucleotide are analyzed using PCR. For example, PCR may be performed using one primer complementary to the inserted nucleotide sequence and another primer complementary to the gene of interest, but outside of the target sequence. The PCR product may then be run on a gel and determination of the expected size of the PCR fragment allows confirmation that the targeting polynucleotide has been inserted in-frame at the desired location. In an exemplary embodiment, host cells are subjected to such a PCR analysis shortly after introduction of the targeting polynucleotide in order to assess correct insertion of the targeting polynucleotide (e.g., insertion of the targeting polynucleotide into the correct target sequence and/or in frame insertion at the target site). The host cells are then grown to dilute out the cells containing essential genes which have been disrupted by the targeting polynucleotide. The cells are then subjected to another round of PCR amplification to detect those cells which no longer produce the desired PCR product. The absence of the desired PCR product in the second round of amplification is indicative that the disrupted gene was essential to growth or viability of the host cell.

In another embodiment, the composition of the amplified tag sequences is analyzed by hybridizing the amplified tags to a high-density oligonucleotide array containing all tag sequences in the population. Methods for making oligonucleotide arrays useful in the present invention are well known in the art (see, e.g., Fodor et al., 1991 Science 251:767-73; Pease et al. 1994 Proc. Natl. Acad. Sci. USA 91:5022-26; Chee et al. 1996 Science 274:610-4; Lipshutz et al. 1995 BioTechniques 19:442-7). The arrays can contain thousands of oligonucleotides (for example, oligonucleotides having about 10-1000 nucleotides, or about 10-100, 10-50, or about 20 nucleotides) representing the set of molecular tags in the total starting cell population. The tag for each of the different strains in the culture hybridizes to a known location on the array, thus facilitating identification of the specific strains that, with increasing culture time or drug concentration, exhibited decreasing (or increasing) hybridization signals on the arrays. In this manner, substantially all molecular tags present in the population can be simultaneously identified without the need for cloning or sequencing. Moreover, it is possible to analyze tags from several timepoints taken during the course of the growth study. The amplified tags from each timepoint can be used to calculate the growth rate of their corresponding strains in the pool.

Amplified sequences can be labeled by, for example, incorporation of a labeled nucleotide (e.g., a fluorescent nucleotide such as Cy3-dUTP or Cy5-dUTP, or a radioactive nucleotide). Labeling can be accomplished by adding detectably labeled nucleotides to a standard PCR reaction containing the appropriate primers. Unincorporated labeled nucleotides are removed (e.g., by size exclusion chromatography) prior to analysis. Alternatively, the amplified tags can be labeled by virtue of a label bound to a common primer used during amplification.

Hybridization of the labeled sequences to the microarray may be accomplished according to methods well known in the art. In exemplary embodiments, hybridization is carried out under conditions that allow for specific hybridization of the amplified tags to their respective complementary sequence located on the array without significant non-specific cross-hybridization. For example, where the molecular tags are about 20 nucleotides in length, hybridization may be carried out in a hybridization mixture of 6×SSPE-T (0.9 M NaCl, 60 mM NaH₂ PO₄, 6 mM EDTA, and 0.005% Triton X-100) for 20 min at 37° C., followed by 10 washes in 1×SSPE-T at 22° C. Following hybridization, the microarrays can be scanned to detect hybridization of the amplified molecular tags using a commercially available detector (for example, the Complete GeneChip™ Instrument System from Affymetrix, Inc., Santa Clara, Calif.) or a custom built scanning laser microscope as described in Shalon et al., (1996) Genome Res. 6:639.

The relative intensity of the hybridization signals for each tag may be determined according to methods well known in the art. For example, the relative hybridization signals can be compared qualitatively to identify strains that are depleted from the sample relative to the other strains. In an exemplary embodiment, when using a diploid strain, a heterozygous disrupted strain can be compared to a non-disrupted homozygous strain to determine the effects of disrupting a single copy of the gene on the growth rate and/or viability of the host cell. Where multiple disrupted strains are produced, the relative hybridization signals of the strains can be compared across samples to identify strains that become under- or over-represented with, for example, increasing culture time or increasing test compound concentration. Where quantitative results are desired, the relative hybridization signal intensities for each strain can be compared over time with the hybridization signal intensities of a control strain (e.g., an undisrupted or “wildtype” strain).

In-frame disrupted strains that become underrepresented with an increasing culture time (or with increasing test compound concentration) relative to an undisrupted strain (or a heterozygote strain that becomes underrepresented relative to an undisrupted diploid strain) are indicative of target genes that confers a selective growth advantage under the growth conditions being tested. For example, where the growth conditions include the presence of a growth inhibiting compound, depletion of a disrupted strain as compared to a wildtype strain indicates that target sequence encodes a gene product that confers resistance to the drug.

The use of an oligonucleotide array allows for quantitative, sensitive, and simultaneous screening of large numbers of in-frame disrupted strains. For example, tags amplified from a pool containing 6,200 different strains, at equal abundance, should generate 6,200 hybridization signals of equal intensity on the array. However, depletion of a disrupted strain (e.g., due to essentiality of the target gene or sensitivity to an inhibitory compound) can be detected by a decreased hybridization signal relative to signals of the same strains at earlier timepoints, relative to other disrupted strains, and relative to a reference strain (e.g., a strain containing a molecular tag without disrupting the target sequence of interest). Inclusion of at least one molecular tag to identify each individual disruption strain within a collection of strains in a culture facilitates screening of test compounds in parallel and allows automation of the various methods disclosed herein.

The combination of growth of pooled strains under test conditions and DNA microarrays according to the methods disclosed herein provides a system of target identification that is potentially genome-wide as well as parallel, highly efficient, and sensitive. By utilizing DNA microarrays and PCR amplification of molecular tags, all strains in a population can be detected and identified simultaneously, even when individual strains differ greatly in their relative abundance. Moreover, microarrays allow analysis of large numbers of different sequences (e.g., up to about 400,000 different oligonucleotides on a single chip of about 1 sq. in.), thus enabling rapid analysis of whole genome experiments.

5. Target Sequences

Targeting polynucleotides may be used for in-frame disruption (e.g., by insertion, deletion, or replacement) of any nucleotide sequence of interest. In exemplary embodiments, a target sequence is all or a portion of a gene encoding an RNA or polypeptide. Creation of a disrupted gene produces a disrupted strain that may have altered expression of the gene product encoded by the target sequence (e.g., by altering copy number or otherwise affecting transcription levels). It is not necessary that the function of the product encoded by the target gene be known; rather, the method of the invention can be used to determine whether the encoded unknown gene product plays a role in survival of the strain under a given set of conditions (e.g., presence of drug, increased temperature, nutrient-deficient medium, etc.). Thus, the target genes can encode any of a variety of gene products, including, but not limited to, genes encoding a protein having an enzymatic activity, structural genes (i.e., DNA sequences which encode a protein or peptide product), regulatory genes (i.e., DNA sequences which act as regulatory regions, such as promoters, enhancers, terminators, translational regulatory regions, etc., to affect the level or pattern of gene expression), and DNA sequences that encode a bioactive RNA, such as an antisense RNA (i.e., to provide for inhibition of expression of a host DNA sequence), or structural RNAs (i.e., RNAs with enzymatic activities or binding activities (ribozymes).

The methods disclosed herein are not limited to examination of the role of “essential” genes, i.e., genes that are conventionally thought to be necessary for cell growth under a given condition or set of conditions. Rather, the invention recognizes that the concept of “essential” genes has hindered the discovery of genes with duplicative function or genes in duplicate pathways that can facilitate resistance to drugs that are targeted against “essential” genes. The exquisite sensitivity of the present methods can be used to unmask such “nonessential” genes that encode potential drug targets of interest, thus facilitating the design of drugs that can be used alone or in combination with conventional drugs to minimize selection of resistant strains, reduce the amount of drug or the time of administration necessary to combat disease, and thus provide a means to avoid side effects associated with administration of high dosages or lengthy drug courses (e.g., toxicity to the subject and other side effects).

6. Recombination System

In various embodiments of the invention, homologous recombination occurs in a host cell capable of carrying out homologous recombination. Recombination generally occurs through the activity of one or more polypeptides which form a “recombination system.” In some embodiments the host cell may contain an endogenous recombination system. In other embodiments, the host cell may contain an endogenous recombination system that may be enhanced by one or more exogenous factors that facilitate recombination in the host cell. For example, the host cell may be engineered to express a polypeptide involved in recombination or a recombination facilitating factor may be mixed with a targeting polynucleotide prior to its introduction into the host cell. In still other embodiments, the host cell may be been engineered to comprise a homologous recombination system that is not endogenous to the cell.

In an exemplary embodiment, a host cell comprises a recombination system having one or more polypeptides encoded by the genes selected from the group consisting of the exo, bet and gam genes from phage λ. The gam gene (also referred to as gamma or γ) encodes a protein which inhibits the RecBCD nuclease from degrading linear DNA while the exo and bet (also referred to as beta or β) genes encode proteins involved in homologous recombination. In one embodiment, the homologous recombination system is the phage λ recombinase system comprising the exo, bet and gam genes of phage λ. Still other suitable recombination systems will be known to one of skill in the art.

The “stuffer” fragment of lambda 1059 carries the lambda exo, beta, gamma under the control of the leftward promoter (pL). These genes confer an Spi⁺ phenotype, i.e., the phage is able to grow on recA⁻ strains but is unable to grow on strains that are lysogenic for bacteriophage P2. Since pL is also located on the “stuffer” fragment, the expression of the Spi⁺ phenotype is not affected by the orientation of the “stuffer” between the left and right arms of the vector.

Wild-type members of the Enterobacteriaceae (e.g., Escherichia coli) are typically resistant to genetic exchange following transformation of linear DNA molecules. This is due, at least in part, to the Exonuclease V (Exo V) activity of the RecBCD holoenzyme which rapidly degrades linear DNA molecules following transformation. Production of ExoV has been traced to the recD gene, which encodes the D subunit of the holoenzyme. The RecBCD holoenzyme plays an important role in initiation of RecA-dependent homologous recombination. Upon recognizing a dsDNA end, the RecBCD enzyme unwinds and degrades the DNA asymmetrically in a 5′ to 3′ direction until it encounters a chi (or “X”)-site (consensus 5′-GCTGGTGG-3′) which attenuates the nuclease activity. This results in the generation of a ssDNA terminating near the c site with a 3′-ssDNA tail that is preferred for RecA loading and subsequent invasion of dsDNA for homologous recombination. Accordingly, preprocessing of transforming fragments with a 5′ to 3′ specific ssDNA Exonuclease, such as Lambda (λ) exonuclease (available, e.g., from Boeringer Mannheim) prior to transformation may serve to stimulate homologous recombination in recD⁻ strains by providing ssDNA invasive end for RecA loading and subsequent strand invasion.

The addition sequences encoding chi-sites (consensus 5′-GCTGGTGG-3′) to DNA fragments can serve to both attenuate Exonuclease V activity and stimulate homologous recombination, thereby obviating the need for a recD mutation (see also, Kowalczykowski, et al. (1994) “Biochemistry of a homologous recombination in Escherichia coli,” Microbiol. Rev. 58:401-465 and Jessen, et al. (1998) “Modification of bacterial artificial chromosomes through Chi-stimulated homologous recombination and its application in zebrafish transgenesis.” Proc. Natl. Acad. Sci. 95:5121-5126).

In certain embodiments, chi-sites may be included in the targeting polynucleotides described herein. The use of recombination-stimulatory sequences such as chi is a generally useful approach for increasing the efficiency of homologous recombination in a wide variety of cell types.

Methods to inhibit or mutate analogs of Exo V or other nucleases (such as, Exonucleases I (endA1), III (nth), IV (nfo), VII, and VIII of E. coli) is similarly useful. Inhibition or elimination of such nucleases, or modification of ends of transforming DNA fragments to render them resistant to exonuclease activity has applications in facilitating homologous recombination in a broad range of cell types.

In certain embodiments, a homologous recombination system may comprise one or more endogenous and/or exogenous recombinase proteins. Recombinases are proteins that may provide a measurable increase in the recombination frequency and/or localization frequency between a targeting polynucleotide and a desired target sequence. The most common recombinase is a family of RecA-like recombination proteins all having essentially all or most of the same functions, particularly: (i) the recombinase protein's ability to properly bind to and position targeting polynucleotides on their homologous targets and (ii) the ability of recombinase protein/targeting polynucleotide complexes to efficiently find and bind to complementary endogenous sequences. The best characterized recA protein is from E. coli, in addition to the wild-type protein a number of mutant recA-like proteins have been identified (e.g., recA803). Further, many organisms have recA-like recombinases with strand-transfer activities (e.g., Fugisawa et al., (1985) Nucl. Acids Res. 13: 7473; Hsieh et al., (1986) Cell 44: 885; Hsieh et al., (1989) J. Biol. Chem. 264: 5089; Fishel et al., (1988) Proc. Natl. Acad. Sci. USA 85: 3683; Cassuto et al., (1987) Mol. Gen. Genet. 208: 10; Ganea et al., (1987) Mol. Cell Biol. 7: 3124; Moore et al., (1990) J. Biol. Chem. 19: 11108; Keene et al., (1984) Nucl. Acids Res. 12: 3057; Kimiec, (1984) Cold Spring Harbor Symp. 48:675; Kimeic, (1986) Cell 44: 545; Kolodner et al., (1987) Proc. Natl. Acad. Sci. USA 84 :5560; Sugino et al., (1985) Proc. Natl. Acad, Sci. USA 85: 3683; Halbrook et al., (1989) J. Biol. Chem. 264: 21403; Eisen et al., (1988) Proc. Natl. Acad. Sci. USA 85: 7481; McCarthy et al., (1988) Proc. Natl. Acad. Sci. USA 85: 5854; Lowenhaupt et al., (1989) J. Biol. Chem. 264: 20568, which are incorporated herein by reference. Examples of such recombinase proteins include, for example but not limitation: recA, recA803, uvsX, and other recA mutants and recA-like recombinases (Roca, A. I. (1990) Crit. Rev. Biochem. Molec. Biol. 25: 415), sep1 (Kolodner et al. (1987) Proc. Natl. Acad. Sci. (U.S.A.) 84: 5560; Tishkoff et al. Molec. Cell. Biol. 11: 2593), RuvC (Dunderdale et al. (1991) Nature 354: 506), DST2, KEM1, XRN1 (Dykstra et al. (1991) Molec. Cell. Biol. 11: 2583), STP-alpha/DST1 (Clark et al. (1991) Molec. Cell. Biol. 11: 2576), HPP-1 (Moore et al. (1991) Proc. Natl. Acad. Sci. (U.S.A.) 88: 9067), other eukaryotic recombinases (Bishop et al. (1992) Cell 69: 439; Shinohara et al. (1992) Cell 69: 457); incorporated herein by reference. RecA may be purified from E. coli strains, such as E. coli strains JC12772 and JC15369 (available from A. J. Clark and M. Madiraju, University of California-Berkeley). These strains contain the recA coding sequences on a “runaway” replicating plasmid vector present at a high copy numbers per cell. The recA803 protein is a high-activity mutant of wild-type recA. The art teaches several examples of recombinase proteins, for example, from Drosophila, yeast, plant, human, and non-human mammalian cells, including proteins with biological properties similar to recA (i.e., recA-like recombinases).

In certain embodiments, recombinase protein(s) (prokaryotic or eukaryotic) may be exogenously administered to a host cell simultaneously or contemporaneously (i.e., within about a few hours) with the targeting polynucleotide(s). Such administration is typically done by microinjection, although electroporation, lipofection, and other transfection methods known in the art may also be used. Alternatively, recombinase proteins may be produced in vivo from a heterologous expression cassette in a transfected cell or transgenic cell, such as a transgenic totipotent embryonal stem cell (e.g., a murine ES cell such as AB-1) used to generate a transgenic non-human animal line or a pluripotent hematopoietic stem cell for reconstituting all or part of the hematopoietic stem cell population of an individual. In exemplary embodiments, a heterologous expression cassette may include a modulatable promoter, such as an ecdysone-inducible promoter-enhancer combination, an estrogen-induced promoter-enhancer combination, a CMV promoter-enhancer, an insulin gene promoter, or other cell-type specific, developmental stage-specific, hormone-inducible, or other modulatable promoter construct so that expression of at least one species of recombinase protein from the cassette can by modulated for transiently producing recombinase(s) in vivo simultaneous or contemporaneous with introduction of a targeting polynucleotide into the cell. When a hormone-inducible promoter-enhancer combination is used, the cell must have the required hormone receptor present, either naturally or as a consequence of expression a co-transfected expression vector encoding such receptor.

For making transgenic non-human animals (which include homologously targeted non-human animals) embryonal stem cells (ES cells) are preferred. Murine ES cells, such as AB-1 line grown on mitotically inactive SNL76/7 cell feeder layers (McMahon and Bradley, Cell 62:1073-1085 (1990)) essentially as described (Robertson, E. J. (1987) in Teratocarcinomas and Embryonic Stem Cells: A Practical Approach. E. J. Robertson, ed. (Oxford: IRL Press), p. 71-112) may be used for homologous gene targeting. Other suitable ES lines include, but are not limited to, the E14 line (Hooper et al. (1987) Nature 326: 292-295), the D3 line (Doetschman et al. (1985) J. Embryol. Exp. Morph. 87: 27-45), and the CCE line (Robertson et al. (1986) Nature 323: 445-448). The success of generating a mouse line from ES cells bearing a specific targeted mutation depends on the pluripotence of the ES cells (i.e., their ability, once injected into a host blastocyst, to participate in embryogenesis and contribute to the germ cells of the resulting animal).

The pluripotence of any given ES cell line can vary with time in culture and the care with which it has been handled. The only definitive assay for pluripotence is to determine whether the specific population of ES cells to be used for targeting can give rise to chimeras capable of germline transmission of the ES genome. For this reason, prior to gene targeting, a portion of the parental population of AB-1 cells is injected into C57B1/6J blastocysts to ascertain whether the cells are capable of generating chimeric mice with extensive ES cell contribution and whether the majority of these chimeras can transmit the ES genome to progeny.

7. Homologous Recombination

Homologous recombination (or general recombination) is defined as the exchange of homologous segments anywhere along a length of two DNA molecules. An essential feature of general recombination is that the enzymes responsible for the recombination event can presumably use any pair of homologous sequences as substrates, although some types of sequence may be favored over others. Both genetic and cytological studies have indicated that such a crossing-over process occurs between pairs of homologous chromosomes during meiosis in higher organisms.

Alternatively, in site-specific recombination, exchange occurs at a specific site, as in the integration of phage λ into the E. coli chromosome and the excision of λ DNA from the E. coli chromosome. Site-specific recombination involves specific inverted repeat sequences; e.g. the Cre-loxP and FLP-FRT systems. Within these sequences there is only a short stretch of homology necessary for the recombination event, but not sufficient for it. The enzymes involved in this event generally cannot recombine other pairs of homologous (or nonhomologous) sequences, but act specifically.

Although both site-specific recombination and homologous recombination are useful mechanisms for genetic engineering of DNA sequences, targeted homologous recombination provides a basis for targeting and altering essentially any desired sequence in a duplex DNA molecule, such as targeting a DNA sequence in a chromosome for replacement by another sequence. Site-specific recombination has been proposed as one method to integrate transfected DNA at chromosomal locations having specific recognition sites (O'Gorman et al. (1991) Science 251: 1351; Onouchi et al. (1991) Nucleic Acids Res. 19: 6373). Unfortunately, since this approach requires the presence of specific target sequences and recombinases, its utility for targeting recombination events at any particular chromosomal location is severely limited in comparison to targeted general recombination.

Homologous recombination may be used to create transgenic animals. Transgenic animals are organisms that contain stably integrated copies of genes or gene constructs derived from another species in the chromosome of the transgenic animal. These animals can be generated by introducing cloned DNA constructs of the foreign genes into totipotent cells by a variety of methods, including homologous recombination. Animals that develop from genetically altered totipotent cells contain the foreign gene in all somatic cells and also in germline cells if the foreign gene was integrated into the genome of the recipient cell before the first cell division. Currently methods for producing transgenics have been performed on totipotent embryonic stem cells (ES) and with fertilized zygotes. ES cells have an advantage in that large numbers of cells can be manipulated easily by homologous recombination in vitro before they are used to generate transgenics.

A primary step in homologous recombination is DNA strand exchange, which involves a pairing of a DNA duplex with at least one DNA strand containing a complementary sequence to form an intermediate recombination structure containing heteroduplex DNA (see, Radding, C. M. (1982) Ann. Rev. Genet. 16: 405; U.S. Pat. No. 4,888,274). The heteroduplex DNA may take several forms, including a three DNA strand containing triplex form wherein a single complementary strand invades the DNA duplex (Hsieh et al. (1990) Genes and Development 4: 1951; Rao et al., (1991) PNAS 88:2984)) and, when two complementary DNA strands pair with a DNA duplex, a classical Holliday recombination joint or chi structure (Holliday, R. (1964) Genet. Res. 5: 282) may form, or a double-D loop. Once formed, a heteroduplex structure may be resolved by strand breakage and exchange, so that all or a portion of an invading DNA strand is spliced into a recipient DNA duplex, adding or replacing a segment of the recipient DNA duplex. Alternatively, a heteroduplex structure may result in gene conversion, wherein a sequence of an invading strand is transferred to a recipient DNA duplex by repair of mismatched bases using the invading strand as a template (Genes, 3rd Ed. (1987) Lewin, B., John Wiley, New York, N.Y.; Lopez et al. (1987) Nucleic Acids Res. 15: 5643). Whether by the mechanism of breakage and rejoining or by the mechanism(s) of gene conversion, formation of heteroduplex DNA at homologously paired joints can serve to transfer genetic sequence information from one DNA molecule to another.

The ability of homologous recombination (gene conversion and classical strand breakage/rejoining) to transfer genetic sequence information between DNA molecules makes targeted homologous recombination a powerful method in genetic engineering and gene manipulation.

The ability of cells to incorporate exogenous genetic material into genes residing on chromosomes has demonstrated that some cells (including yeast, mammals and humans) have the general enzymatic machinery for carrying out homologous recombination required between resident and introduced sequences. These targeted recombination events can be used to correct mutations at known sites, replace genes or gene segments with defective ones, or introduce foreign genes into cells. The efficiency of such gene targeting techniques is related to several parameters: the efficiency of DNA delivery into cells, the type of DNA packaging (if any) and the size and conformation of the incoming DNA, the length and position of regions homologous to the target site (all these parameters also likely affect the ability of the incoming homologous DNA sequences to survive intracellular nuclease attack), the efficiency of hybridization and recombination at particular chromosomal sites and whether recombinant events are homologous or nonhomologous.

Over the past 10 years or so, several methods have been developed to introduce DNA into mammalian cells: direct needle microinjection, transfection, electroporation, retroviruses, adenoviruses, adeno-associated viruses; Herpes viruses, and other viral packaging and delivery systems, polyamidoamine dendimers, liposomes, and more recently techniques using DNA-coated microprojectiles delivered with a gene gun (called a biolistics device), or narrow-beam lasers (laser-poration). The processes associated with some types of gene transfer have been shown to be pathogenic, mutagenic or carcinogenic (Bardwell, (1989) Mutagenesis 4: 245), and these possibilities must be considered in choosing a transfection approach.

The choice of a particular DNA transfection procedure depends upon its availability to the researcher, the technique's efficiency with the particular chosen target cell type, and the researchers concerns about the potential for generating unwanted genome mutations. For example, retroviral integration requires dividing cells, most often results in nonhomologous recombination events, and retroviral insertion within a coding sequence of nonhomologous (i.e., non-targeted) gene could cause cell mutation by inactivating the gene's coding sequence (Friedmann, (1989) Science 244:1275). Newer retroviral-based DNA delivery systems are being developed using modified retroviruses. However, these disabled viruses must be packaged using helper systems, are often obtained at low titer, and recombination is still not site-specific, thus recombination between endogenous cellular retrovirus sequences and disabled virus sequences could still produce wild-type retrovirus capable of causing gene mutation. Adeno- or polyoma virus based delivery systems appear promising (Samulski et al., (1991) EMBO J. 10: 2941; Gareis et al., (1991) Cell. Molec. Biol. 37: 191; Rosenfeld et al. (1992) Cell 68: 143) although they still require specific cell membrane recognition and binding characteristics for target cell entry. Liposomes often show a narrow spectrum of cell specificities, and when DNA is coated externally on to them, the DNA is often sensitive to cellular nucleases. Newer polycationic lipospermines compounds exhibit broad cell ranges (Behr et al., (1989) Proc. Natl. Acad. Sci. USA 86: 6982) and DNA is coated by these compounds. In addition, a combination of neutral and cationic lipid has been shown to be highly efficient at transfection of animal cells and showed a broad spectrum of effectiveness in a variety of cell lines (Rose et al., (1991) BioTechniques 10:520). Galactosylated bis-acridine has also been described as a carrier for delivery of polynucleotides to liver cells (Haensler J L and Szoka F C (1992), Abstract V211 in J. Cell. Biochem. Supplement 16F, Apr. 3-16, 1992, incorporated herein by reference). Electroporation also appears to be applicable to most cell types. The efficiency of this procedure for a specific gene is variable and can range from about one event per 3×10⁴ transfected cells (Thomas and Capecchi, (1987) Cell 51: 503) to between one in 10⁷ and 10⁸ cells receiving the exogenous DNA (Koller and Smithies, (1989) Proc. Natl. Acad. Sci. (U.S.A.) 86: 8932). Microinjection of exogenous DNA into the nucleus has been reported to result in stable integration in transfected cells. Zimmer and Gruss (Zimmer and Gruss (1989) Nature 338: 150) have reported that for the mouse hox1.1 gene, 1 per 150 microinjected cells showed a stable homologous site specific alteration.

Several methods have been developed to detect and/or select for targeted site-specific recombinants between vector DNA and the target homologous chromosomal sequence (see, Capecchi, (1989) Science 244: 1288 for review). Cells which exhibit a specific phenotype after site-specific recombination, such as occurs with alteration of the hprt gene, can be obtained by direct selection on the appropriate growth medium. Alternatively, a selective marker sequence such as neo can be incorporated into a vector under promoter control, and successful transfection can be scored by selecting G418^{r} cells followed by PCR to determine whether neo is at the targeted site (Joyner et al., (1989) Nature 338: 153). A positive-negative selection (PNS) procedure using both neo and HSV-tk genes allows selection for transfectants and against nonhomologous recombination events, and significantly enriched for desired disruption events at several different mouse genes (Mansour et al., (1988) Nature 336: 348). This procedure has the advantage that the method does not require that the targeted gene be transcribed. If the targeted gene is transcribed, a promoter-less marker gene can be incorporated into the targeting construct so that the gene becomes activated after homologous recombination with the target site (Jasin and Berg, (1988) Genes and Development 2: 1353; Doetschman et al. (1988) Proc. Natl. Acad. Sci. (U.S.A.) 85: 8583; Dorini et al., (1989) Science 243: 1357; Itzhaki and Porter, (1991) Nucl. Acids Res. 19: 3835). Recombinant products produced using vectors with selectable markers often continue to retain these markers as foreign genetic material at the site of transfection, although loss does occur. Valancius and Smithies (Valancius and Smithies, (1991) Mole. Cellular Biol. 11: 1402) have described an “in-out” targeting procedure that allowed a subtle 4 bp insertion modification of a mouse hprt target gene. The resulting transfectant contained only the desired modified gene sequence and no selectable marker remained after the “out” recombination step. Cotransformation of cells with two different vectors, one vector contained a selectable gene and the other used for gene disruption, increases the efficiency of isolating a specific targeting reaction (Reid et al., (1991) Molec. Cellular Biol. 11: 2769) among selected cells that are subsequently scored for stable recombinants.

Unfortunately, exogenous sequences transferred into eukaryotic cells undergo homologous recombination with homologous endogenous sequences only at very low frequencies, and are so inefficiently recombined that large numbers of cells must be transfected, selected, and screened in order to generate a desired correctly targeted homologous recombinant (Kucherlapati et al. (1984) Proc. Natl. Acad. Sci. (U.S.A.) 81: 3153; Smithies, O. (1985) Nature 317: 230; Song et al. (1987) Proc. Natl. Acad. Sci. (U.S.A.) 84: 6820; Doetschman et al. (1987) Nature 330: 576; Kim and Smithies (1988) Nucleic Acids Res. 16: 8887; Doetschman et al. (1988) op.cit.; Koller and Smithies (1989) op.cit.; Shesely et al. (1991) Proc. Natl. Acad. Sci. (U.S.A.) 88: 4294; Kim et al. (1991) Gene 103: 227, which are incorporated herein by reference).

Koller et al. (1991) Proc. Natl. Acad. Sci. (U.S.A.), 88: 10730 and Snouwaert et al. (1992) Science 257: 1083, have described targeting of the mouse cystic fibrosis transmembrane regulator (CFTR) gene for the purpose of inactivating, rather than correcting, a murine CFTR allele. Koller et al. employed a large (7.8 kb) homology region in the targeting construct, but nonetheless reported a low frequency for correct targeting (only 1 of 2500 G418-resistant cells were correctly targeted). Thus, even targeting constructs having long homology regions are inefficiently targeted.

Several proteins or purified extracts having the property of promoting homologous recombination (i.e., recombinase activity) have been identified in prokaryotes and eukaryotes (Cox and Lehman (1987) Ann. Rev. Biochem. 56: 229; Radding, C. M. (1982) op.cit.; Madiraju et al. (1988) Proc. Natl. Acad. Sci. (U.S.A.) 85: 6592; McCarthy et al. (1988) Proc. Natl. Acad. Sci. (U.S.A.) 85: 5854; Lopez et al. (1987) op.cit., which are incorporated herein by reference). These general recombinases presumably promote one or more steps in the formation of homologously-paired intermediates, strand-exchange, gene conversion, and/or other steps in the process of homologous recombination.

The frequency of homologous recombination in prokaryotes is significantly enhanced by the presence of recombinase activities. Several purified proteins catalyze homologous pairing and/or strand exchange in vitro, including: E. coli recA protein, the T4 uvsX protein, the rec1 protein from Ustilago maydis, and Rad51 protein from S. cervisiae —(Sung et al., Science 265:1241 (1994)) and human cells (Baumann et al., Cell 87:757 (1996)). Recombinases, like the recA protein of E. coli are proteins which promote strand pairing and exchange. The most studied recombinase to date has been the recA recombinase of E. coli, which is involved in homology search and strand exchange reactions (see, Cox and Lehman (1987) op.cit.). RecA is required for induction of the SOS repair response, DNA repair, and efficient genetic recombination in E. coli. RecA can catalyze homologous pairing of a linear duplex DNA and a homologous single strand DNA in vitro. In contrast to site-specific recombinases, proteins like recA which are involved in general recombination recognize and promote pairing of DNA structures on the basis of shared homology, as has been shown by several in vitro experiments (Hsieh and Camerini-Otero (1989) J. Biol. Chem. 264: 5089; Howard-Flanders et al. (1984) Nature 309: 215; Stasiak et al. (1984) Cold Spring Harbor Symp. Quant. Biol. 49: 561; Register et al. (1987) J. Biol. Chem. 262: 12812). Several investigators have used recA protein in vitro to promote homologously paired triplex DNA (Cheng et al. (1988) J. Biol. Chem. 263: 15110; Ferrin and Camerini-Otero (1991) Science 354: 1494; Ramdas et al. (1989) J. Biol Chem. 264: 11395; Strobel et al. (1991) Science 254: 1639; Hsieh et al. (1990) op.cit.; Rigas et al. (1986) Proc. Natl. Acad. Sci. (U.S.A.) 83: 9591; and Camerini-Otero et al. U.S. Pat. No. 7,611,268 (available from Derwent), which are incorporated herein by reference).

Common mechanisms for inducing recombination include, but are not limited to the use of strains comprising mutations such as those involved in mismatch repair. e.g. mutations in mutS, mutT, mutL and mutH; exposure to U.V. light; Chemical mutagenesis, e.g. use of inhibitors of MMR, DNA damage inducible genes, or SOS inducers; overproduction/underproduction/mutation of any component of the homologous recombination complex/pathway, eg. RecA, ssb, etc.; overproduction/underproduction/mutation of genes involved in DNA synthesis/homeostasis; overproduction/underproduction/mutation of recombination-stimulating genes from bacteria, phage (eg. Lambda Red function), or other organisms; addition of chi sites into/flanking the donor DNA fragments; coating the DNA fragments with RecA/ssb and the like.

8. Exemplary Targets

The methods of the invention can be used in connection with any of a variety of host cells, including eukaryotic, prokaryotic, diploid, or haploid organisms. In various embodiments, host cells may be single cell organisms (e.g., bacteria, e.g., Mycobacterium spp., e.g., M. tuberculosis) or may be derived from multicellular organisms (transgenic organisms, such as insects (e.g., Drosophila), worms (e.g., Caenorhabditis spp, e.g., C. elegans) and higher animals (e.g., transgenic mammals such as mice, rats, rabbits, hamsters, etc.). In certain embodiments, the host cell is a naturally diploid cell, preferably yeast cells (e.g., Saccharomyces spp. (e.g., S. cerevisiae), Candida spp. (e.g., C. albicans)) or mammalian cells (e.g., mouse, monkey, or human). The host cell may also be a cell infected with a virus or phage that contains a target sequence in the viral or phage genome. In exemplary embodiments, part or all of the genome of the host organism has been sequenced. Host cells may be naturally competent for transformation or may be made competent for transformation.

Examples of disease causing viruses that may be used in accord with the methods described herein include: Retroviridae (e.g., human immunodeficiency viruses, such as HIV-1 (also referred to as HTLV-III, LAV or HTLV-III/LAV, See Ratner, L. et al., Nature, Vol. 313, Pp. 227-284 (1985); Wain Hobson, S. et al, Cell, Vol. 40: Pp. 9-17 (1985)); HIV-2 (See Guyader et al., Nature, Vol. 328, Pp. 662-669 (1987); European Patent Publication No. 0 269 520; Chakraborti et al., Nature, Vol. 328, Pp. 543-547 (1987); and European Patent Application No. 0 655 501); and other isolates, such as HIV-LP (International Publication No. WO 94/00562 entitled “A Novel Human Immunodeficiency Virus”; Picornaviridae (e.g., polio viruses, hepatitis A virus, (Gust, I. D., et al., Intervirology, Vol. 20, Pp. 1-7 (1983); entero viruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses′); Poxviridae (variola viruses, vaccinia viruses, pox viruses); and Iridoviridae (e.g., African swine fever virus); and unclassified viruses (e.g., the etiological agents of Spongiform encephalopathies, the agent of delta hepatities (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Examples of infectious bacteria include: Helicobacter pylori, Borrelia burgdorferi, Legionella pneumophilia, Mycobacterium sps. (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, and Actinomyces israelli.

Examples of infectious fungi include: Cryptococcus neoformans, Histoplasma capsulatum, Coccidioides immitis, Blastomyces dermatitidis, Chlamydia trachomatis, Candida albicans. Other infectious organisms (i.e., protists) include: Plasmodium falciparum and Toxoplasma gondii.

Genomic information (including nucleotide sequences, amino acid sequences, protein expression information, and/or protein structure information) for a variety of microorganisms may be found in the databases maintained by The Institute for Genomic Research (TIGR) (www.tigr.org) and/or the National Center for Biotechnology Information (NCBI) (www.ncbi.nlm.nih.gov). Examples of bacteria for which genomic information is available, include, for example, Agrobacterium tumefaciens str. C58 (Cereon) (NC_{—}003062 & NC_{—}003063), Agrobacterium tumefaciens str. C58 (U. Washington) (NC_{—}003304 & NC_{—}003305), Aquifex aeolicus (NC_{—}000918), Bacillus halodurans (NC_{—}002570), Bacillus subtilis (NC_{—}000964), Borrelia burgdorferi (NC_{—}001318), Brucella melitensis (NC_{—}003317 & NC_{—}003318), Buchnera sp. APS (NC_{—}002528), Campylobacter jejuni (NC_{—}002163), Caulobacter crescentus —CB15 (NC_{—}002696), Chlamydia muridarum (NC_{—}002620), Chlamydia trachomatis (NC_{—}000117), Chlamydophila pneumoniae AR39 (NC_{—}002179), Chlamydophila pneumoniae CWL029 (NC_{—}000922), Chlamydophila pneumoniae J138 (NC_{—}002491), Clostridium acetobutylicum (NC_{—}003030), Clostridium perfringens (NC_{—}003366), Corynebacterium glutamicum (NC_{—}003450), Deinococcus radiodurans (NC_{—}001263 & NC_{—}001264), Escherichia coli K12 (NC_{—}000913), Escherichia coli O157:H7 (NC_{—}002695), Escherichia coli O157:H7 EDL933 (NC_{—}002655), Fusobacterium nucleatum subsp. nucleatum ATCC 25586 (NC_{—}003454), Haemophilus influenzae Rd (NC_{—}000907), Helicobacter pylori 26695 (NC_{—}000915), Helicobacter pylori J99 (NC_{—}000921), Lactococcus lactis subsp. lactis (NC_{—}002662), Listeria innocua (NC_{—}003212), Listeria monocytogenes EGD-e (NC_{—}003210), Mesorhizobium loti (NC_{—}002678), Mycobacterium leprae (NC_{—}002677), Mycobacterium tuberculosis CDC1551 (NC_{—}002755), Mycobacterium tuberculosis H37Rv (NC_{—}000962), Mycoplasma genitalium (NC_{—}000908), Mycoplasma pneumoniae (NC_{—}000912), Mycoplasma pulmonis (NC_{—}002771), Neisseria meningitidis MC58 (NC_{—}003112), Neisseria meningitidis (NC_{—}003116), Nostoc sp. (NC_{—}003272), Pasteurella multocida (NC_{—}002663), Pseudomonas aeruginosa (NC_{—}002516), Ralstonia solanacearum (NC_{—}003295 & NC_{—}003296), Rickettsia conorii (NC_{—}003103), Rickettsia prowazekii (NC_{—}000963), Salmonella enterica subsp. enterica serovar Typhi (NC_{—}003198), Salmonella typhi (NC_{—}002305), Salmonella typhimurium LT2 (NC_{—}003197), Sinorhizobium meliloti (NC_{—}003047), Staphylococcus aureus subsp. aureus MW2 (NC_{—}003923), Staphylococcus aureus subsp. aureus Mu50 (NC_{—}002758), Staphylococcus aureus subsp. aureus N315 (NC_{—}002745), Streptococcus pneumoniae R6 (NC_{—}003098), Streptococcus pneumoniae TIGR4 (NC_{—}003028), Streptococcus pyogenes M1 GAS (NC_{—}002737), Streptococcus pyogenes MGAS8232 (NC_{—}003485), Streptomyces coelicolor A3(2) (NC_{—}003888), Synechocystis sp. PCC 6803 (NC_{—}000911), Thermoanaerobacter tengcongensis (NC_{—}003869), Thermotoga maritima (NC_{—}000853), Treponema pallidum (NC_{—}000919), Ureaplasma urealyticum (NC_{—}002162), Vibrio cholerae (NC_{—}002505 & NC_{—}002506), Xanthomonas axonopodis pv. citri str. 306 (NC_{—}003919), Xanthomonas campestris pv. campestris str. ATCC 33913 (NC_{—}003902), Xylella fastidiosa 9a5c (NC_{—}002488), and Yersinia pestis (NC_{—}003143).

Examples of archaea for which genomic information is available from TIGR and/or NCBI, include, for example, Aeropyrum pernix (NC_{—}000854), Archaeoglobus fulgidus (NC_{—}000917), Halobacterium sp. NRC-1 (NC_{—}002607), Methanococcus jannaschii (NC_{—}000909), Methanopyrus kandleri AV19 (NC_{—}003551), Methanosarcina acetivorans str. C2A (NC_{—}003552), Methanosarcina mazei Goe1 (NC_{—}003901), Methanothermobacter thermautotrophicus (NC_{—}000916), Pyrobaculum aerophilum (NC_{—}003364), Pyrococcus abyssi (NC_{—}000868), Pyrococcus furiosus DSM 3638 (NC_{—}003413), Pyrococcus horikoshii (NC_{—}000961), Sulfolobus solfataricus (NC_{—}002754), Sulfolobus tokodaii (NC_{—}003106), Thermoplasma acidophilum (NC_{—}002578), and Thermoplasma volcanium (NC_{—}002689).

Examples of eukaryotes for which genomic information is available from TIGR and/or NCBI, include, for example, Anopheles gambiae, Arabidopsis thaliana, Caenorhabditis elegans, Drosophila melanogaster, Encephalitozoon cuniculi, Guillardia theta nucleomorph, Saccharomyces cerevisiae, and Schizosaccharomyces pombe.

Genomic information for over 900 viral species is available from TIGR and/or NCBI, including, for example, information about deltaviruses, retroid viruses, satellites, dsDNA viruses, dsRNA viruses, ssDNA viruses, ssRNA negative-strand viruses, ssRNA positive-strand viruses, unclassified bacteriophages, and other unclassified viruses.

Proteome information (including amino acid sequences, amino acid composition, protein families, protein domains, structural information and/or functional information) for a variety of microorganisms may be found in the Proteome Analysis Database maintained by the European Bioinformatics Institute (EBI) (http://www.ebi.ac.uk/proteome). Examples of bacteria for which proteome information is available, include, for example, Agrobacterium tumefaciens strain c58 (Cereon), Agrobacterium tumefaciens strain c58 (U. Washington), Anabaena sp. strain PCC 7120, Aquifex aeolicus, Bacillus halodurans, Bacillus subtilis, Borrelia burgdorferi, Brucella melitensis, Buchnera aphidicola (subsp. Acyrthosiphon pisum), Campylobacter jejuni, Caulobacter crescentus, Chlamydia muridarum, Chlamydia pneumoniae strain AR39, Chlamydia pneumoniae strain CWL029, Chlamydia pneumoniae strain J138, Chlamydia trachomatis, Clostridium acetobutylicum, Clostridium perfringens, Deinococcus radiodurans, Escherichia coli K-12, Escherichia coli O157:H7 strain EDL933, Escherichia coli O157:H7 substrain RIMD 0509952, Haemophilus influenzae, Helicobacter pylori strain 26695, Helicobacter pylori strain J99, Lactococcus lactis (subsp. lactis) strain IL1403, Listeria innocua, Listeria monocytogenes, Mycobacterium leprae, Mycobacterium tuberculosis strain H37Rv, Mycoplasma genitalium, Mycoplasma pneumoniae, Mycoplasma pulmonis, Neisseria meningitidis strain Z2491 (serogroup A), Neisseria meningitidis strain MC58 (serogroup B), Pasteurella multocida, Pseudomonas aeruginosa, Ralstonia solanacearum, Rhizobium meliloti, Rhizobium loti, Rickettsia conorii, Rickettsia prowazekii, Salmonella typhi, Salmonella typhimurium, Staphylococcus aureus strain Mu50, Staphylococcus aureus strain N315, Streptococcus pneumoniae strain TIGR4, Streptococcus pyogenes strain SF370, Synechocystis sp. PCC 6803, Thermotoga maritima, Treponema pallidum, Ureaplasma parvum, Vibrio cholerae, Xylella fastidiosa, and Yersinia pestis.

Examples of archaea for which proteome information is available from EBI, include, for example, Aeropyrum pernix K1, Archaeoglobus fulgidus, Halobacterium sp. NRC-1, Methanococcus jannaschii, Methanobacterium thermoautotrophicum, Pyrobaculum aerophilum, Pyrococcus abyssi, Pyrococcus horikoshi, Sulfolobus solfataricus, Sulfolobus tokodaii, Thermoplasma acidophilum, and Thermoplasma volcanium.

Examples of eukaryotes for which proteome information is available from EBI, include, for example, Arabidopsis thaliana, Caenorhabditis elegans, Drosophila melanogaster, Guillardia theta (algal nucleomorph), Homo sapiens, Mus musculus, Saccharomyces cerevisiae, and Schizosaccharomyces pombe.

In another embodiment, eukaryotic cells may be used as a host cell for homologous recombination. For making transgenic non-human animals (which include homologously targeted non-human animals) embryonal stem cells (ES cells) and fertilized zygotes are exemplary. In one embodiment, embryonal stem cells may be used as a host cell for homologous gene targeting, such as murine ES cells like the AB-1 line that may be grown on mitotically inactive SNL76/7 cell feeder layers (McMahon and Bradley, Cell 62: 1073-1085 (1990)) essentially as described (Robertson, E. J. (1987) in Teratocarcinomas and Embryonic Stem Cells: A Practical Approach. E. J. Robertson, ed. (oxford: IRL Press), p. 71-112). Other suitable ES lines include, but are not limited to, the E14 line (Hooper et al. (1987) Nature 326: 292-295), the D3 line (Doetschman et al. (1985) J. Embryol. Exp. Morph. 87: 21-45), and the CCE line (Robertson et al. (1986) Nature 323: 445-448). The success of generating a mouse line from ES cells bearing a specific targeted mutation depends on the pluripotence of the ES cells (i.e., their ability, once injected into a host blastocyst, to participate in embryogenesis and contribute to the germ cells of the resulting animal).

The pluripotence of any given ES cell line can vary with time in culture and the care with which it has been handled. The only definitive assay for pluripotence is to determine whether the specific population of ES cells to be used for targeting can give rise to chimeras capable of germline transmission of the ES genome. For this reason, prior to gene targeting, a portion of the parental population of AB-1 cells is injected into C57B1/6J blastocysts to ascertain whether the cells are capable of generating chimeric mice with extensive ES cell contribution and whether the majority of these chimeras can transmit the ES genome to progeny.

In another embodiment, non-human zygotes may be used, for example to make transgenic animals, using techniques known in the art (see U.S. Pat. No. 4,873,191). Preferred zygotes include, but are not limited to, animal zygotes, including fish, avian and mammalian zygotes. Suitable fish zygotes include, but are not limited to, those from species of salmon, trout, tuna, carp, flounder, halibut, swordfish, cod, tulapia and zebrafish. Suitable bird zygotes include, but are not limited to, those of chickens, ducks, quail, pheasant, turkeys, and other jungle fowl and game birds. Suitable mammalian zygotes include, but are not limited to, cells from horses, cows, buffalo, deer, sheep, rabbits, rodents such as mice, rats, hamsters and guinea pigs, goats, pigs, primates, and marine mammals including dolphins and whales. See Hogan et al., Manipulating the Mouse Embryo (A Laboratory Manual), 2nd Ed. Cold Spring Harbor Press, 1994, incorporated by reference.

9. Biological Drug Screening Assays

The compositions and methods described herein are useful for identifying genes encoding products that are suitable candidates for targeting by therapeutic and diagnostic agents. For example, genes identified in accordance with this method as essential to a selected pathogen in the infection process, and proteins encoded thereby, may serve as targets for the screening and development of natural or synthetic chemical compounds which have utility as therapeutic drugs for the treatment of infection by this pathogen. As an example, a compound capable of binding to protein encoded by an essential gene and inhibiting its biological activity may be useful as a drug component to preventing diseases or disorders resulting from the growth of a particular organism. Alternatively, compounds which inhibit expression or reduce expression of an essential gene are also believed to be useful therapeutically.

Conventional assays and techniques may be used for screening and development of such therapeutics. For example, a method for identifying compounds which specifically bind to or inhibit proteins encoded by these gene sequences can include simply the steps of contacting a selected protein or gene product with a test compound to permit binding of the test compound to the protein; and determining the amount of test compound, if any, which is bound to the protein. Such a method may involve the incubation of the test compound and the protein immobilized on a solid support. Still other conventional methods of drug screening can involve employing a suitable computer program to determine compounds having similar or complementary structure to that of the gene product or portions thereof and screening those compounds for competitive binding to the protein. Such compounds may be incorporated into an appropriate therapeutic formulation, alone or in combination with other active ingredients. Methods of formulating such therapeutic compositions, as well as suitable pharmaceutical carriers, and the like are well known to those of skill in the art.

Accordingly, through use of such methods, the present invention is believed to provide compounds capable of interacting with the products of essential or conditionally essential genes, or fragments thereof, and either enhancing or decreasing the biological activity, as desired. Such compounds are also encompassed by this invention.

For therapeutic uses, the compounds, compositions, or agents identified using the methods disclosed herein may be administered systemically, for example, formulated in a pharmaceutically-acceptable buffer such as physiological saline. Treatment may be accomplished directly, e.g., by treating the animal with antagonists which disrupt, suppress, attenuate, or neutralize the biological events associated with a pathogen. Preferable routes of administration include, for example, inhalation or subcutaneous, intravenous, interperitoneally, intramuscular, or intradermal injections which provide continuous, sustained levels of the drug in the patient. Treatment of human patients or other animals will be carried out using a therapeutically effective amount of an anti-bacterial agent in a physiologically-acceptable carrier. Suitable carriers and their formulation are described, for example, in Remington's Pharmaceutical Sciences by E. W. Martin. The amount of the anti-bacterial agent to be administered varies depending upon the manner of administration, the age and body weight of the patient, and with the type of disease and extensiveness of the disease. Generally, amounts will be in the range of those used for other agents used in the treatment of other microbial diseases, although in certain instances lower amounts will be needed because of the increased specificity of the compound. A compound is administered at a dosage that inhibits microbial proliferation or survival. For example, for systemic administration a compound is administered typically in the range of 0.1 ng-10 g/kg body weight.

For agricultural uses, the compounds, compositions, or agents identified using the methods disclosed herein may be used as chemicals applied as sprays or dusts on the foliage of plants, or in irrigation systems. Typically, such agents are to be administered on the surface of the plant in advance of the pathogen in order to prevent infection. Seeds, bulbs, roots, tubers, and corms are also treated to prevent pathogenic attack after planting by controlling pathogens carried on them or existing in the soil at the planting site. Soil to be planted with vegetables, ornamentals, shrubs, or trees can also be treated with chemical fumigants for control of a variety of microbial pathogens. Treatment is preferably done several days or weeks before planting. The chemicals can be applied by either a mechanized route, e.g., a tractor or with hand applications. In addition, chemicals identified using the methods of the assay can be used as disinfectants.

In addition, the antipathogenic agent may be added to materials used to make catheters, including but not limited to intravenous, urinary, intraperitoneal, ventricular, spinal and surgical drainage catheters, in order to prevent colonization and systemic seeding by potential pathogens. Similarly, the antipathogenic agent may be added to the materials that, constitute various surgical prostheses and to dentures to prevent colonization by pathogens and thereby prevent more serious invasive infection or systemic seeding by pathogens.

### EQUIVALENTS

The present invention provides among other things novel polynucleotides and methods of use thereof. While specific embodiments of the subject invention have been discussed, the above specification is illustrative and not restrictive. Many variations of the invention will become apparent to those skilled in the art upon review of this specification. The appended claims are not intended to claim all such embodiments and variations, and the full scope of the invention should be determined by reference to the claims, along with their full scope of equivalents, and the specification, along with such variations.

### INCORPORATION BY REFERENCE

All publications and patents mentioned herein, including those items listed below, are hereby incorporated by reference in their entirety as if each individual publication or patent was specifically and individually indicated to be incorporated by reference. In case of conflict, the present application, including any definitions herein, will control. Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

Also incorporated by reference are the following: Yu et al. (2000) Proc. Natl. Acad. Sci. 97(11): 5978-5983; Datsenko & Wanner (2000) Proc. Natl. Acad. Sci. 97(12): 6640-6645; Shoemaker, et al. (1996) Nature Genetics 14: 450-456; Rothstein et al., Meth. Enzymol 194: 281 (1991); Berinstein et al., Molec. Cell. Biol. 12: 360 (1992); U.S. Pat. Nos. 6,139,817; 6,207,384; 6,255,113; 6,150,160; 6,200,812; 6,251,674; 6,046,002; WO 99/35256; WO 98/12352; WO 99/35494; WO 99/53079; and EP 0967291; and GenBank Accession numbers NP_{—}040616, P03697, AAA96569, BAB19617, NP_{—}049473, AAF28114, AAD25418, P03698, NP_{—}040617, NC_{—}001416, AAA96517, and J02459.

We claim:

1. A method for disrupting a gene of an organism without introducing downstream effects, comprising:
(a) providing a host cell of an organism, wherein the host cell is capable of homologous recombination;
(b) identifying an open reading frame for a gene of the organism;
(c) introducing into the host cell a targeting polynucleotide comprising (i) a molecular tag and (ii) flanking homology clamps for aligning the targeting polynucleotide in-frame with the open reading frame of the gene; and
(d) selecting host cells having an in-frame disruption of the gene.

2. The method of claim 1, wherein the host cell comprises an endogenous recombination system.

3. The method of claim 1, wherein the host cell is expressing the exo, bet and gam genes from phage λ.

4. The method of claim 1, which further comprises passage of the cells.

5. The method of claim 1, further comprising characterizing the essentiality of the gene by determining whether the in-frame insertion of the targeting polynucleotide into the genome of the host cell produced a change in the phenotype of the host cell attributable to disruption of the gene in the absence of any downstream effects.

6. The method of claim 5, wherein the change in the phenotype of the host cell is a change in the growth rate or viability of the host cell.

7. The method of claim 6, wherein a change in the growth rate or viability of the host cell is determined using PCR or DNA microarray analysis.

8. The method of claim 5, wherein the change in the phenotype of the host cell occurs under an environmental condition selected from the group consisting of oxygen tension, osmolarity, pH, temperature, nutrient availability, prolonged stationary phase, presence of a test compound, conditions encountered in a disease state, and combinations thereof.

9. The method of claim 1, wherein the molecular tag is inserted into the genome of the host cell under the control of the transcriptional, translational, or transcriptional and translational regulatory sequences of the disrupted gene.

10. The method of claim 1, wherein host cells having in-frame disruptions are selected by PCR with one primer that hybridizes to a region within the targeting polynucleotide insertion and one primer that hybridizes to a region of the genome of the host cell outside of the disrupted gene sequence.

11. The method of claim 1, wherein the molecular tag is a reporter gene encoding for a selectable or detectable marker.

12. The method of claim 11, wherein host cells having in-frame disruptions are selected based on expression of the reporter gene.

13. The method of claim 1, wherein the host cell is haploid.

14. The method of claim 1, wherein the host cell is diploid.

15. The method of claim 14, wherein disruption of one copy of a gene produces a change in the phenotype of the host cell.

16. The method of claim 1, wherein the host cell is prokaryotic.

17. The method of claim 16, wherein the host cell is selected from the group consisting of bacteria and archaebacteria.

18. The method of claim 1, wherein the host cell is eukaryotic.

19. The method of claim 18, wherein the eukaryotic cell is selected from the group consisting of algae, yeast, fungus, plant, insect, reptile, amphibian, fish, bird, rodent, mammal, monkey and human.

20. A method for characterizing the essentiality of a plurality of genes from an organism by in-frame gene disruption, comprising:
(a) providing a host cell of an organism, wherein the host cell is capable of homologous recombination;
(b) identifying at least five open reading frames for at least five genes of the organism;
(c) introducing into the host cell a targeting polynucleotide for each of the genes comprising (i) a molecular tag and (ii) flanking homology clamps for aligning the targeting polynucleotides in-frame with the open reading frames of the genes;
(d) selecting host cells having in-frame disruptions of the genes; and
(e) determining whether the in-frame insertions of the targeting polynucleotides into the genome of the host cell produced a change in the phenotype of the host cell attributable to disruption of the genes in the absence of any downstream effects.

21. The method of claim 20, wherein at least 25 genes are targeted for disruption by at least 25 targeting polynucleotides.

22. The method of claim 21, wherein at least 50 genes are targeted for disruption by at least 50 targeting polynucleotides.

23. The method of claim 22, wherein at least 100 genes are targeted for disruption by at least 100 targeting polynucleotides.

24. The method of claim 23, wherein at least 500 genes are targeted for disruption by at least 500 targeting polynucleotides.

25. The method of claim 20, wherein the at least 5 targeting polynucleotides are introduced into a single host cell culture.

26. The method of claim 20, wherein the at least 5 targeting polynucleotides are introduced into separate cultures of host cells.

27. The method of claim 20, wherein the sequence for each of the at least 5 molecular tags is the same.

28. The method of claim 20, wherein the sequence for each of the at least 5 molecular tags is different.

29. The method of claim 20, wherein all of the at least 5 targeting polynucleotides are introduced into the genome in-frame.

30. A database comprising the identity of essential genes for an organism wherein substantially all of the genes have been disrupted in-frame using the method of claim 1.
